(19) United States
(12) Patent Application Publication
(10) Pub. No.: US 2018/0080891 A1
(43) Pub. Date: Mar. 22, 2018

(54) SYSTEMS AND METHODS FOR ENVIRONMENT SENSING

(71) Applicant: General Electric Company, Schenectady, NY (US)

(72) Inventors: Radislav Alexandrovich Potyrailo, Niskayuna, NY (US); Nasr Alkadi, Oklahoma City, OK (US)

(21) Appl. No.: 15/271,692

(22) Filed: Sep. 21, 2016

(51) Int. Cl. G01N 27/04; G01N 33/00

(52) U.S. Cl. CPC G01N 27/04; G01N 33/0036

(57) ABSTRACT

Systems and methods are provided for environment sensing. The system includes a sensor having a sensing material and at least one pair of electrodes in contact with the sensing material, the sensing material configured to be in contact with an ambient environment. The system includes a controller circuit electrically coupled to the at least one pair of electrodes. The controller circuit is configured to generate a stimulation waveform for application to the sensing material of the sensor via the at least one pair of electrodes. The controller circuit is configured to receive an electrical signal from the at least one pair of electrodes representative of an impedance response of the sensing material, and analyze the impedance response of the sensing material at frequencies that provide a linear response of the sensing material to an analyte of interest and at least partially reject effects of interferences.

### FIELD

Embodiments of the subject matter disclosed herein generally relate to systems and methods for environment sensing.

### BACKGROUND

Environmental sensors, such as metal-oxide sensors, are often employed in a number of applications where the detection of various vapors or gases may be used to discern useful information. For example, environmental sensors may be utilized to monitor industrial areas for chemical or physical hazards, such as the detection of carbon dioxide within the chemical manufacturing industry, detection of engine exhaust gases such as carbon monoxide, hydrocarbons, and nitrogen oxides within the transportation industry, and detection of fugitive methane emissions within the oil and gas industry.

One technique for sensing such environmental changes is by employing a conventional sensor, such as a radio frequency identification (RFID) sensor, a resistance sensor, and/or a capacitance sensor coated with a particular sensing material. The impedance, resistance, capacitance response of the conventional sensor can be measured via inductive coupling or directly by connecting to a sensor reader. The electrical response of the conventional sensor is translated into the impedance, resistance, or capacitance changes of the conventional sensor, which is utilized to determine a concentration of a chemical vapor of interest, such as carbon dioxide, carbon monoxide, and nitrogen oxide, or methane gas. However, available conventional sensors suffer from a non-linear response, specifically an exponential, power law, and/or non-monotonic response, as a function of the chemical vapor concentration. Due to the power law response, as the concentration of the chemical vapor increases, the chemical vapor saturates the conventional sensor response leading to significant errors in estimation of the chemical vapor concentration. The terms “gas” and “vapor” describe any volatile species that are in contact with the sensor.

Additionally, conventional sensors are affected by other chemical vapors (e.g., not the chemical vapor of interest) exposed to the conventional sensor, such as a concentration of water vapor (e.g., ambient humidity). The water vapor shifts or saturates the response of the conventional sensor, which can affect a determination of the concentration of the chemical vapor of interest by the sensor.

### BRIEF DESCRIPTION

In an embodiment, a method (e.g., for detecting an analyte of interest) includes receiving a stimulation waveform at a sensor. The stimulation waveform is applied to a sensing material of the sensor via at least one pair of electrodes in contact with the sensing material. The sensing material is in contact with an ambient environment. The method includes receiving an electrical signal at a controller circuit from the at least one pair of electrodes representative of an impedance response of the sensing material, and analyzing the impedance response of the sensing material at frequencies that provide a linear response of the sensing material to an analyte of interest and at least partially rejects effects of interferences.

In an embodiment, a system (e.g., sensing system) includes a sensor having a sensing material and at least one pair of electrodes in contact with the sensing material, the sensing material configured to be in contact with an ambient environment. The system includes a controller circuit electrically coupled to the at least one pair of electrodes. The controller circuit is configured to generate a stimulation waveform for application to the sensing material of the sensor via the at least one pair of electrodes. The controller circuit is configured to receive an electrical signal from the at least one pair of electrodes representative of an impedance response of the sensing material, and analyze the impedance response of the sensing material at frequencies that provide a linear response of the sensing material to an analyte of interest and at least partially reject effects of interferences.

In an embodiment, a method (e.g., for detecting one or more analytes of interest) includes receiving a stimulation waveform at a sensor. The stimulation waveform is applied to a sensing material of the sensor via at least one pair of electrodes in contact with the sensing material. The sensing material is in contact with an ambient environment. The method includes receiving an electrical signal at a controller circuit from the at least one pair of electrodes representative of an impedance response of the sensing material, and analyzing the impedance response of the sensing material at frequencies that provide a monotonic or non-monotonic response of the sensing material to an analyte of interest and at least partially reject effects of interferences.

### BRIEF DESCRIPTION OF THE DRAWINGS

The presently described subject matter will be better understood from reading the following description of non-limiting embodiments, with reference to the attached drawings, wherein below:

FIG. 1 is a schematic diagram of one embodiment of a sensing system;

FIG. 2 is a schematic diagram of one embodiment of a sensing system;

FIG. 3 shows a graphical illustration of one embodiment of a stimulation waveform applied to a sensing material of a sensor;

FIG. 4A shows graphical illustrations of a measured response corresponding to a non-resonance impedance response of a sensor, in accordance with an embodiment;

FIG. 4B shows graphical illustrations of a measured response corresponding to a resonance impedance response of a sensor, in accordance with an embodiment;

FIG. 5 is a flow chart of one embodiment of a method for detecting one or more analytes of interest;

FIG. 6 is a graphical illustration of a spectral parameter calculated from a conventional sensor;

FIG. 7 is a graphical illustration of a concentration curve of the conventional sensor based on the spectral parameter shown in FIG. 6;

FIG. 8 is a graphical illustration of one embodiment of a spectral parameter calculated from a sensor;

FIG. 9 is a graphical illustration of a concentration curve of the sensor based on the spectral parameter shown in FIG. 8;

FIG. 10 is a graphical illustration of a typical effect of an analyte of interest and ambient humidity on a spectral parameter of a conventional sensor;

FIG. 11 is a graphical illustration of one embodiment of an analyte of interest and ambient humidity on a spectral parameter of a sensor;

FIG. 12 is a graphical illustration of one embodiment of a spectral parameter of one embodiment of a sensor, in accordance with an embodiment;

FIG. 13 is a graphical illustration of one embodiment of a principal components analysis of a plurality of spectral parameters;

FIG. 14 are graphical illustrations of spectral parameters of one embodiment of a measured response of a sensor;

FIG. 15 are graphical illustrations of spectral parameters of one embodiment of a measured response of a sensor;

FIG. 16 is a graphical illustration of a spectral parameter calculated from a conventional sensor; and

FIG. 17 is a graphical illustration of a spectral parameter of an embodiment calculated from a sensor.

### DETAILED DESCRIPTION

One or more embodiments herein describe systems and methods for environment sensing, specifically for detecting one or more analytes of interest within an environment. Exemplary existing and emerging applications of sensors include environmental monitoring and protection, industrial safety and manufacturing process control, monitoring of agricultural emissions, public safety, medical systems, wearable health and fitness, automation of residential homes and industrial buildings, transportation, and retail. Examples of classes and types of measured gases and volatiles of interest for these applications include environmental background (e.g. O₂, CO₂, H₂O), transportation/industrial/agricultural atmospheric pollutants (e.g. CO₂, CO, O₃, H₂S, NH₃, NOₓ, SO₂, CH₄, industrial fumes, waste odors), breath biomarkers (e.g. NO, H₂S, NH₄, acetone, ethane, pentane, isoprene, hydrogen peroxide), and public/homeland safety hazardous volatiles (e.g. toxic industrial chemicals, chemical warfare agents, explosives). Diverse types of volatiles are needed to be monitored over their broad range of concentrations ranging from part-per-trillion to percent, often mixed with chemical interferences such as ubiquitous variable background (indoor and outdoor urban air, industrial air, human odors and breath, exhaust of transportation engines, etc.), and at expected operation temperatures (ambient indoor and outdoor temperatures, body temperature, exhaust of transportation engines). Various embodiments utilize a sensing material electrically coupled to a pair of electrodes. An electrical stimulus is delivered to the metal oxide sensor or a transducer that includes a sensing material. Optionally, the sensor may include a resonant inductor L-capacitor C-resistor R (LCR) circuit and/or an RFID sensor.

An impedance response (e.g., impedance spectrum) of the sensor is measured via a controller circuit directly and/or inductive coupled between a pick up coil and the sensor. For example, the electrical response at certain frequencies or a single frequency corresponding to signal changes (e.g., impedance, resistance, capacitance, and/or the like) of the sensor is translated into the impedance changes of the sensor to form the impedance response. Based on the impedance response, the controller circuit may calculate one or more spectrum parameters. The “spectrum” or “spectral” parameters are calculated from a real portion and/or imaginary portion of the impedance response. The spectrum parameters are utilized to determine an environmental parameter of the analyte of interest. For example, the controller circuit may analyze the impedance response of the sensing material at frequencies calculated from the real portion of the impedance response that provide a linear response of the sensing material to the analyte of interest. It may be noted, the impedance response of the sensing material described herein provides a linearity improvement over the nonlinear (e.g. power law) resistance response of the sensing material in conventional environmental sensors. Additionally due to the linear response, the impedance response of the sensing material provides a monotonic response improvement over the non-monotonic resistance response (e.g., parabolic) of the sensing material in conventional environmental sensors. Additionally or alternatively, the spectrum parameters may be selected to reject and/or filter out effects of interference due to volatile analytes (e.g., analytes not of interest). For example, the impedance response of the sensing material provides reduction of effects of humidity over the resistance response of the sensing material in conventional environmental sensors.

Optionally, the sensors described herein may be utilized in a wireless sensor network as described in U.S. patent application entitled, “SYSTEMS AND METHODS FOR ENVIRONMENT SENSING” having docket number 312706-1US, which is incorporated by reference in its entirety.

The fluids described herein can include gases, vapors, liquids, particles, biological particles, and/or biological molecules. Optionally, a fluid may refer to one or more solid materials.

A digital identification or ID can include data stored in a memory chip (or other memory device) of an RFID sensor. Non-limiting examples of this data include manufacturer identification, electronic pedigree data, user data, and/or calibration data for the sensor.

A monitoring process includes, but is not limited to, measuring physical changes that occur around the sensor. For example, monitoring processes including monitoring changes in a biopharmaceutical, food or beverage manufacturing process related to changes in physical, chemical, and/or biological properties of an environment around the sensor. Monitoring processes may also include those industry processes that monitor physical changes as well as changes in a component's composition or position. Non-limiting examples include homeland security monitoring, residential home protection monitoring, environmental monitoring, clinical or bedside patient monitoring, airport security monitoring, admission ticketing, and other public events. Monitoring can be performed when the sensor signal has reached an appreciably steady state response and/or when the sensor has a dynamic response. The steady state sensor response is a response from the sensor over a determined period of time, where the response does not appreciably change over the measurement time. Thus, measurements of steady state sensor response over time produce similar values. The dynamic sensor response is a response from the sensor upon a change in the measured environmental parameter (temperature, pressure, chemical concentration, biological concentration, etc.). Thus, the dynamic sensor response significantly changes over the measurement time to produce a dynamic signature of response toward the environmental parameter or parameters measured. Non-limiting examples of the dynamic signature of the response include average response slope, average response magnitude, largest positive slope of signal response, largest negative slope of signal response, average change in signal response, maximum positive change in signal response, and maximum negative change in signal response. The produced dynamic signature of response can be used to further enhance the selectivity of the sensor in dynamic measurements of individual vapors and their mixtures. The produced dynamic signature of response can also be used to further optimize the combination of sensing material and transducer geometry to enhance the selectivity of the sensor in dynamic and steady state measurements of individual vapors and their mixtures.

Environmental parameters and/or select parameters can refer to measurable environmental variables within or surrounding a manufacturing or monitoring system (e.g., a sensing system). The measurable environmental variables comprise at least one of physical, chemical, and biological properties and include, but are not limited to, measurement of temperature, pressure, material concentration, conductivity, dielectric property, number of dielectric, metallic, chemical, or biological particles in the proximity or in contact with the sensor, dose of ionizing radiation, and light intensity.

An analyte can include any desired measured environmental parameter.

Interference includes an undesired environmental parameter that undesirably affects the accuracy and precision of measurements with the sensor. An interference includes a fluid or an environmental parameter (that includes, but is not limited to temperature, pressure, light, etc.) that potentially may produce an interference response by the sensor.

A multivariate analysis can refer to a mathematical procedure that is used to analyze more than one variable from the sensor response and to provide the information about the type of at least one environmental parameter from the measured sensor spectral parameters and/or to quantitative information about the level of at least one environmental parameter from the measured sensor spectral parameters. A principal components analysis (PCA) includes a mathematical procedure that is used to reduce multidimensional data sets to lower dimensions for analysis. Principal component analysis is a part of eigenanalysis methods of statistical analysis of multivariate data and may be performed using a covariance matrix or correlation matrix. Non-limiting examples of multivariate analysis tools include canonical correlation analysis, regression analysis, nonlinear regression analysis, principal components analysis, discriminate function analysis, multidimensional scaling, linear discriminate analysis, logistic regression, or neural network analysis.

Spectral parameters or spectrum parameters may be used to refer to measurable variables of the impedance response of the sensor. The impedance sensor response is the impedance spectrum of the non-resonance sensor circuit of the CR (capacitance (C)-resistance (R)) sensor. The impedance sensor response is the impedance spectrum of the resonance sensor circuit of the LCR (inductance (L)-capacitance (C)-resistance (R)) or RFID (radio-frequency identification) sensor. In addition to measuring the impedance spectrum in the form of Z-parameters, S-parameters, and other parameters, the impedance spectrum (both real and imaginary parts) may be analyzed simultaneously using various parameters for analysis, such as, the frequency of the maximum of the real part of the impedance (Fp), the magnitude of the real part of the impedance (Zp), the resonant frequency of the imaginary part of the impedance (F1), and the anti-resonant frequency of the imaginary part of the impedance (F2), signal magnitude (Z1) at the resonant frequency of the imaginary part of the impedance (F1), signal magnitude (Z2) at the anti-resonant frequency of the imaginary part of the impedance (F2), and zero-reactance frequency (Fz, frequency at which the imaginary portion of impedance is zero). Other spectral parameters may be simultaneously measured using the entire impedance spectra, for example, quality factor of resonance, phase angle, and magnitude of impedance. Collectively, “spectral parameters” or “spectrum parameters” calculated from the impedance spectra (such as non-resonance or resonance spectra), are called here “features” or “descriptors.” The appropriate selection of features is performed from all potential features that can be calculated from spectra. Multivariable spectral parameters are described in U.S. Pat. No. 7,911,345 entitled “Methods and systems for calibration of RFID sensors,” which is incorporated herein by reference.

A resonance impedance or impedance may refer to measured sensor frequency response from which the sensor spectral parameters are extracted.

Sensing materials and/or sensing films may include, but are not limited to, materials deposited onto a transducer's electronics module, such as electrodes of the CR or LCR circuit components or an RFID tag, to perform the function of predictably and reproducibly affecting the impedance sensor response upon interaction with the environment. For example, a conducting polymer such as polyaniline changes its conductivity upon exposure to solutions of different pH. When such a polyaniline film is deposited onto the CR or the LCR or RFID sensor, the impedance sensor response changes as a function of pH. Thus, such as a CR or LCR or RFID sensor works as a pH sensor. When such a polyaniline film is deposited onto the CR or LCR or RFID sensor for detection in gas phase, the impedance sensor response also changes upon exposure to basic (for example, NH₃) or acidic (for example, HCl) gases. Alternatively, the sensing film may be a dielectric polymer. Sensor films include, but are not limited to, polymer, organic, inorganic, biological, composite, and nano-composite films that change their electrical and or dielectric property based on the environment that they are placed in. Non-limiting additional examples of sensor films may be a sulfonated polymer such as Nafion, an adhesive polymer such as silicone adhesive, an inorganic film such as sol-gel film, a composite film such as carbon black-polyisobutylene film, a nanocomposite film such as carbon nanotube-Nafion film, gold nanoparticle-polymer film, metal nanoparticle-polymer film, electrospun polymer nanofibers, electrospun inorganic nanofibers, electrospun composite nanofibers, or films/fibers doped with organic, metallorganic or biologically derived molecules and any other sensing material. In order to prevent the material in the sensor film from leaching into the liquid environment, the sensing materials are attached to the sensor surface using standard techniques, such as covalent bonding, electrostatic bonding, and other standard techniques known to those of ordinary skill in the art. In addition, the sensing material has at least two temperature-dependent response coefficients related to temperature-dependent changes in material dielectric constant and resistance of the sensing material.

Transducer and/or sensor may be used to refer to electronic devices such as CR, LCR or RFID devices intended for sensing. Transducer can be a device before it is coated with a sensing film or before it is calibrated for a sensing application. A sensor may be a device typically after it is coated with a sensing film and after being calibrated for the sensing application.

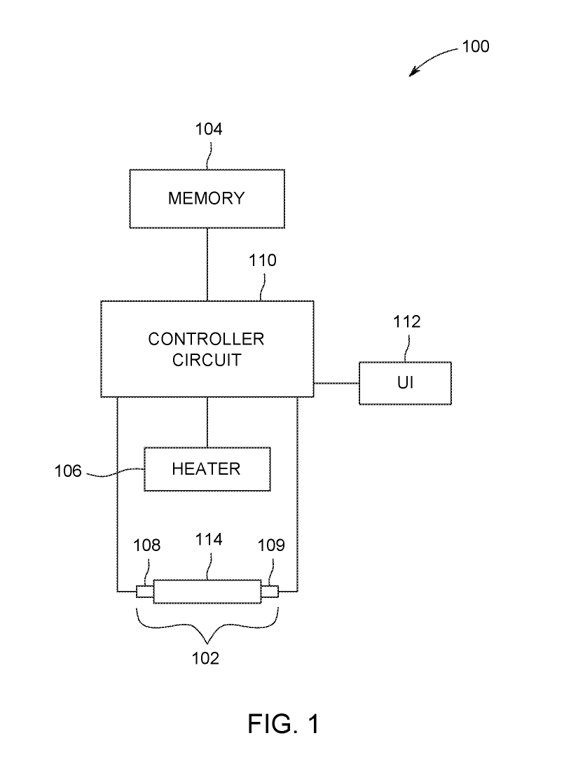

FIG. 1 is a schematic diagram of a sensing system 100, in accordance with an embodiment. The sensing system 100 includes a controller circuit 110, a memory 104, a heater 106, and a sensor 102. The memory 104 is an electronic storage device configured to store information acquired from the sensor 102 (e.g., an impedance spectrum, a transfer function, and/or the like). The contents of the memory 104 may be accessed by the controller circuit 110, and/or the like. The memory 104 may include flash memory, RAM, ROM, EEPROM, and/or the like.

The controller circuit 110 may control the operation of the sensing system 100. For example, the controller circuit 110 may be configured to apply a stimulation waveform to the sensor 102. The stimulation waveform may be an electrical stimulus configured to be a sinusoidal waveform having an amplitude (e.g., voltage, current, and/or the like) and a dynamic frequency. Optionally, the controller circuit 110 may adjust the frequency of the stimulation waveform over time. For example, the controller circuit 110 may adjust the frequency of the stimulation waveform between frequencies of a non-resonant bandwidth of the sensor 102. In another example, the stimulation waveform may adjust the frequency of the stimulation waveform between frequencies of a scanning bandwidth of the sensor 102. The scanning bandwidth includes a range of frequencies that are non-resonant frequencies of the sensor 102.

The controller circuit 110 is configured to acquire an impedance response of the sensor 102 in response to the stimulation waveform. For example, the controller circuit 110 analyzes the impedance response of the sensor 102 at frequencies that provide a linear response within a predetermined threshold (e.g., sufficiently linear) of the sensor 102 to the one or more analytes of interest. The controller circuit 110 may also be configured to analyze the impedance response of the sensor 102 at frequencies that provide a non-linear response, monotonic response or a non-monotonic response within a predetermined threshold of the sensor 102 to the one or more analytes of interest. The controller circuit 110 may be embodied in hardware, such as a processor, controller, or other logic-based device, that performs functions or operations based on one or more sets of instructions (e.g., software). The instructions on which the hardware operates may be stored on a tangible and non-transitory (e.g., not a transient signal) computer readable storage medium, such as the memory 104. Alternatively, one or more of the sets of instructions that direct operations of the hardware may be hard-wired into the logic of the hardware.

The heater 106 may be thermally coupled to the sensor 102. The heater 106 is configured to generate thermal energy. For example, the heater 106 may include one or more heating elements configured to convert electrical power (e.g., current, voltage) to generate thermal energy (e.g., heater). The amount of thermal energy generated by the heater 106 may be based on instructions received by the controller circuit 110. The thermal energy generated by the heater 106 is received by the sensor 102, and may increase a temperature of the sensor 102 above an ambient temperature of the environment. For example, the heater 106 may increase a temperature of the sensor 102 at least 50 degrees Celsius above the ambient temperature. Additionally or alternatively, the heater 106 may generate thermal energy to raise a temperature of the sensor 102 based on a predetermined temperature stored in the memory 104 (e.g., known to be above ambient temperature). For example, the controller circuit 110 may instruct the heater 106 to increase a temperature of the sensor 102 to at least 100 degrees Celsius. In another example, the controller circuit 110 may instruct the heater 106 to increase a temperature of the sensor 102 to at least 200 degrees Celsius. In yet another example, the controller circuit 110 may instruct the heater 106 to increase a temperature of the sensor 102 to at least 800 degrees Celsius.

The heater 106 may be a part of an asset or a part of equipment and may be thermally coupled to the sensor 102. For example, the heater 106 may be an internal combustion engine, a turbine, a gas stack, a chemical reactor vessel, a melting vessel and the like.

Optionally, the sensing system 100 may include a user interface 112. The user interface 112 may correspond to a switch, a relay, a tactile button, and/or the like. The user interface 112 may be used by the controller circuit 110 to receive a user input to determine when to generate a stimulation waveform. In another example, the user interface 112 may be used by the controller circuit 110 to determine when to calibrate the sensor, such as defining a transfer function of the sensor 102. Additionally or alternatively, the user interface 112 may include one or more visual and/or audio indicators configured to alert a status of the sensing system 100 to the user.

The sensor 102 is configured to measure and/or detect a presence of one or more analytes of interest within the ambient (e.g., in operational contact with the sensing material 114, proximate to, surrounding area, within a predetermined distance of a surface are of the sensing material 114, and/or the like) environment of the sensor 102. The sensor 102 includes at least one pair of electrodes 108-109 and a sensing material 114. The electrodes 108-109 are conductors that are electrically coupled to the sensing material 114 and the controller circuit 110. For example, the electrodes 108-109 are in contact with the sensing material 114. The electrodes 108-109 are configured to deliver the stimulation waveform generated by the controller circuit 110 to the electrodes 108-109 and to the sensing material 114.

The sensing material 114 is configured to predictably and reproducibly affect and adjust the impedance of the sensor 114 in response to changes in the environment. For example, characteristics (e.g., magnitude of the real part of the impedance, magnitude of the imaginary part of the impedance, phase of the impedance, and/or the like) of the impedance of the sensing material 114 are adjusted based on a concentration, presence, and/or the like of the analyte of interest within the ambient environment of the sensor 102. The sensing material 114 is in operational contact with the ambient environment. For example, at least a portion of a surface area of the sensing material 114 is exposed to and/or in contact with the environment adjacent to the sensor 102, which changes an electrical property (e.g., inductance) of the sensing material 114. The sensing material 114 may be a semiconducting polymer (e.g., polyaniline film, Nafion) and/or a dielectric polymer (e.g., silicone adhesive). Additionally or alternatively, the sensing material 114 may include organic, inorganic (e.g., sol-gel film), biological, composite film (e.g., polyisobutylene film), a nano-composite film (e.g., electrospun polymer nanofibers, gold nanoparticle-polymer film, metal nanoparticle-polymer film, electrospun polymer nanofibers, electrospun inorganic nanofibers, electrospun composite nanofibers), n-type oxide semiconductor, p-type oxide semiconductor, graphene, carbon nanotubes, and/or the like that are configured to change an electrical and/or dielectric property based on an environment exposed to the sensing material 114.

Additionally or alternatively, the sensing material 114 may be a metal oxide. For example, the sensing material 114 may be a single-metal oxide such as ZnO, CuO, CoO, SnO2, TiO2, ZrO2, CeO2, WO3, MoO3, In2O3, and/or the like. In another example, the sensing material 114 may be a perovskite oxide having differently sized cations such as SrTiO3, CaTiO3, BaTiO3, LaFeO3, LaCoO3, SmFeO3, and/or the like. In another example, the sensing material 114 may be a mixed metal oxide composition such as CuO—BaTiO3, ZnO—WO3, and/or the like.

Base sensing materials may be further doped with metal salts, metal nanoparticles, conducting nanoparticles, semiconducting nanoparticles. Morphology of the base sensing materials may influence the working temperature of the sensing material. Sensing materials may be used for detection of analyte gases at a temperature of at least 30 degrees Celsius. Non-limiting examples of such sensing materials may include CeO, Fe2O3, In2O3, WO3, GaAs, SnO2, ZnO, NiO, V2O5, and/or the like.

Sensing materials may be used for detection of analyte gases at a temperature of at least 100 degrees Celsius. Non-limiting examples of such sensing materials may include LaCoO3, GaAs. Sensing materials may be used for detection of analyte gases at a temperature of at least 300 degrees Celsius. Non-limiting examples of such sensing materials may include ZnO, AlVO4, SnO2, Bi4Fe2O9, La2CuO4, WO3, and/or the like. Sensing materials may be used for detection of analyte gases at a temperature of at least 800 degrees Celsius. Non-limiting examples of such sensing materials may include BaTiO3, SrTiO3, Ga2O3, WO3, Nb2O3, MoO3, CeO2, BaSnO3, and/or the like. Such sensing materials with the associated sensors may detect gases and volatiles of environmental background (e.g. O2, CO2, H2O), transportation/industrial/agricultural atmospheric pollutants (e.g. CO2, CO, O3, H2S, NH3, NOx, SO2, CH4, industrial fumes, waste odors), breath biomarkers (e.g. NO, H2S, NH4, acetone, ethane, pentane, isoprene, hydrogen peroxide), and public/homeland safety hazardous volatiles (e.g. toxic industrial chemicals, chemical warfare agents, explosives).

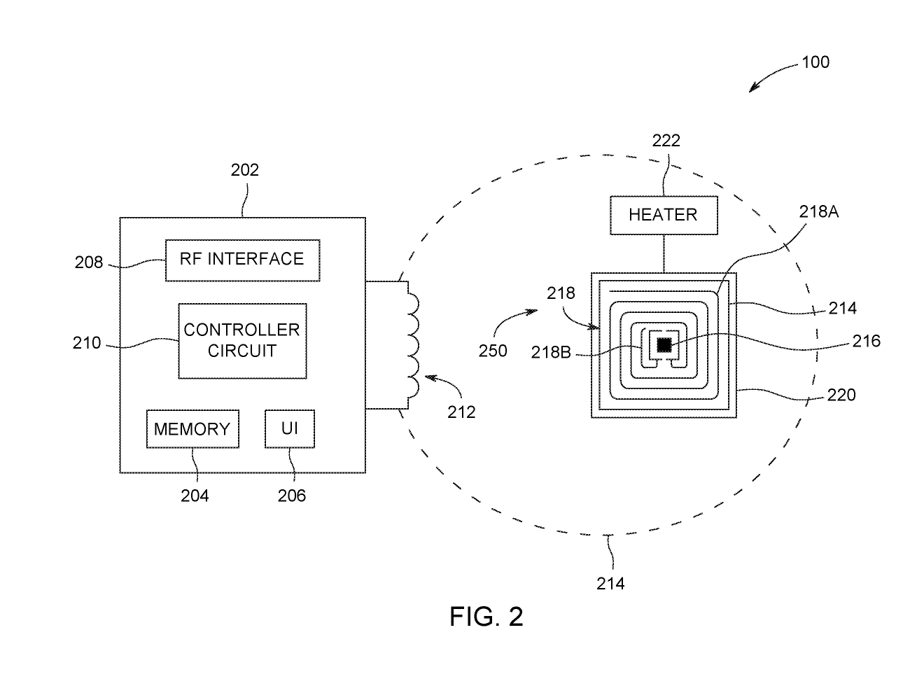

The sensor 102 may be configured as a non-resonant circuit. Additionally or alternatively, the sensor 102 may be configured as a resonant circuit by adding one or more components (e.g., inductor). Optionally, in connection with FIG. 2, a sensing system 200 may include a sensor 250 configured as a resonant circuit. It may be noted, that the sensor 250 may be configured as a resonant circuit which may be implemented as the sensor 102.

FIG. 2 is a schematic diagram of the sensing system 200, in accordance with an embodiment. The sensing system 200 is having sensor reader 202 and a sensor 250. The sensor reader 202 may include a memory 204, a radio frequency (RF) interface 208, an antenna 212, and a controller circuit 210. The sensor reader 202 may be configured to receive an impedance of the sensor 250, for example via a mutual inductance coupling between the sensor 250 and a pickup coil 212 of the sensor reader 202. Optionally, the sensor reader 202 may include a user interface 206.

The RF interface 208 may be electrically coupled to the memory 204, the controller circuit 210, and the pickup coil 212. The RF interface 208 may include a transmitter, a receiver, a transmitter and a receiver (e.g., a transceiver), and/or the like. The RF interface 208 may be configured to transmit and/or receive information using an RFID protocol. The RFID protocol may be a short range wireless communication protocol defined in ISO/IEC 18092/ECMA-340, ISO/IEC 18000, ISO/IEC 14443, and/or the like. The RF interface 208 may include hardware, such as a processor, controller, or other logic-based device to conform and/or encode information stored in the memory 204 to the RFID protocol to transmit using the pickup coil 212, and/or decode information received by the pickup coil 212 to be processed by the RF interface 208 and/or the controller circuit 210.

The memory 204 is an electronic storage device configured to store information received from the sensor 250 (e.g., an impedance, a transfer function, and/or the like). The contents of the memory 204 may be accessed by the controller circuit 210, the RF interface 208, and/or the like. The memory 204 may include flash memory, RAM, ROM, EEPROM, and/or the like.

The controller circuit 210 may control the operation of the sensor reader 202. The controller circuit 210 may be embodied in hardware, such as a processor, controller, or other logic-based device, that performs functions or operations based on one or more sets of instructions (e.g., software). The instructions on which the hardware operates may be stored on a tangible and non-transitory (e.g., not a transient signal) computer readable storage medium, such as the memory 204. Alternatively, one or more of the sets of instructions that direct operations of the hardware may be hard-wired into the logic of the hardware.

The user interface 206 may include a switch, a relay, a tactile button, and/or the like. The user interface 206 may be used by the RF interface 208 to determine when to receive information from and/or transmit information to the sensor 250.

The sensor 250 is configured to detect the one or more analytes of interest. Optionally, the sensor 250 may be similar to the sensors described in U.S. Pat. No. 9,037,418 entitled “Highly selective chemical and biological sensors,” U.S. Pat. No. 8,542,024 entitled “Temperature-independent chemical and biological sensors, and U.S. Publication No. 2012/0235690 entitled “Methods for analyte detection,” all of which are incorporated by reference in their entirety. The sensor 250 may include a heater 222 thermally coupled to the sensing material 214. The heater 222 may be similar to and/or the same as the heater 106.

The sensor 250 includes a resonant inductor capacitor resistor (LCR) circuit with a sensing material 214 overlaid on a substrate 220. The resonant LCR circuit is formed and/or defined by a sensor antenna 218 (e.g., 218a-b). The sensor antenna 218 may be divided into at least one pair of electrodes, such as a first electrode (e.g., the sensor antenna 218a) and a second electrode (e.g., the sensor antenna 218b). Additionally or alternatively, the sensor antenna 218 may be a single electrode, such as a single conducting structure operationally coupled to a substrate. The sensing material 214 is disposed and/or applied over a sensing region of the substrate 220, which is interposed between the sensor antenna 218. For example, the sensing material 214 is attached to the sensor region of the substrate 220 by covalent bonding, electrostatic bonding and/or the like. The sensing material 214 material may be similar to and/or the same as the sensing material 114 shown in FIG. 1.

Additionally or alternatively (not illustrated), a complementary sensor may be attached across the antenna 218 that does not have the controller circuit 216 and alters sensor impedance response. For example, the complementary sensor may be interdigitated sensor, resistive sensor, and capacitive sensor, and/or the like. Complementary sensors are described in U.S. Pat. No. 7,911,345 entitled “Methods and systems for calibration of RFID sensors,” which is incorporated herein by reference.

Optionally, the sensor 250 may also include a controller circuit 216 electrically coupled to the antenna 218. The controller circuit 216 may be configured to apply the stimulation waveform to the antenna 218. The controller circuit 216 may include a memory and an RF signal modulation circuitry. The memory may include manufacturing, user, calibration, a transfer function, and/or other data stored thereon. The controller circuit 216 may be an integrated circuit fabricated using a complementary metal-oxide semiconductor (CMOS) process and a non-volatile memory. The controller circuit 216 may include an analog I/O input utilized for example as a resistance input, capacitance input, inductance input, and/or the like. The RF signal modulation circuitry may include a diode rectifier, a power supply voltage control, a modulator, a demodulator, a clock generator, and other components.

The sensor 250 may be communicatively coupled to the sensor reader 202 enabling the controller circuit 216 to read (e.g., accessed) and/or store information received by the sensor reader 202 via the antenna 218. For example, the memory of the controller circuit 216 may be read wirelessly by the sensor reader 202 using a mutual inductance coupling between the antenna 218 and the pickup coil 212. The pickup coil 212 may be positioned within an activation field 214 of the antenna 218. For example, an alternating current passes within the pickup coil 212 to generate an RF and/or microwave field, which is passed through the antenna 218. Optionally, the current may pass within the pickup coil 212 in response to a user input received by the user interface 206. The activation field 214 may correspond to a region from the antenna 218 where an RF and/or microwave field generated by the pickup coil 112 can be received by the antenna 218. A size of the activation field 214 may be based on a frequency of the RF and/or microwave field, a power level and/or amplitude of the RF and/or microwave field, a size (e.g., dimensions, length, width, and/or the like) of the antenna 218, and/or the like. An AC voltage is generated across the antenna 218 based on the RF and/or microwave field emitted by the pickup coil 212, which is rectified by the controller circuit 216 via the RF signal modulation circuitry to result in a DC voltage for the operation of the sensor 250 to form the mutual inductance coupling. Additionally or alternatively, the sensor 250 may include a power source (not shown) that may be used to generate power (e.g., current, voltage) for the operation of the sensor 250. Additionally or alternatively, the sensor 250 may be communicatively coupled to the sensor reader 202 via a wired interface.

Optionally, the AC voltage generated across the antenna 218 may correspond to the stimulation waveform utilized to measure the impedance response. For example, sensing is performed via monitoring of the changes in the electrical properties (e.g., to form the impedance response) of the sensing material 214 as probed by the electromagnetic field generated at the antenna 218 in response to the RF and/or microwave field emitted by the sensor reader 202. Upon reading the sensor 250 with the pickup coil 212, the electromagnetic field generated in the antenna 218 extends out from the plane of the sensor 250 and is affected by the dielectric property of a sensing material that is in contact with an ambient environment that adjusts an electrical characteristic to enable the controller circuit 110 to measure the one or more analytes of interest.

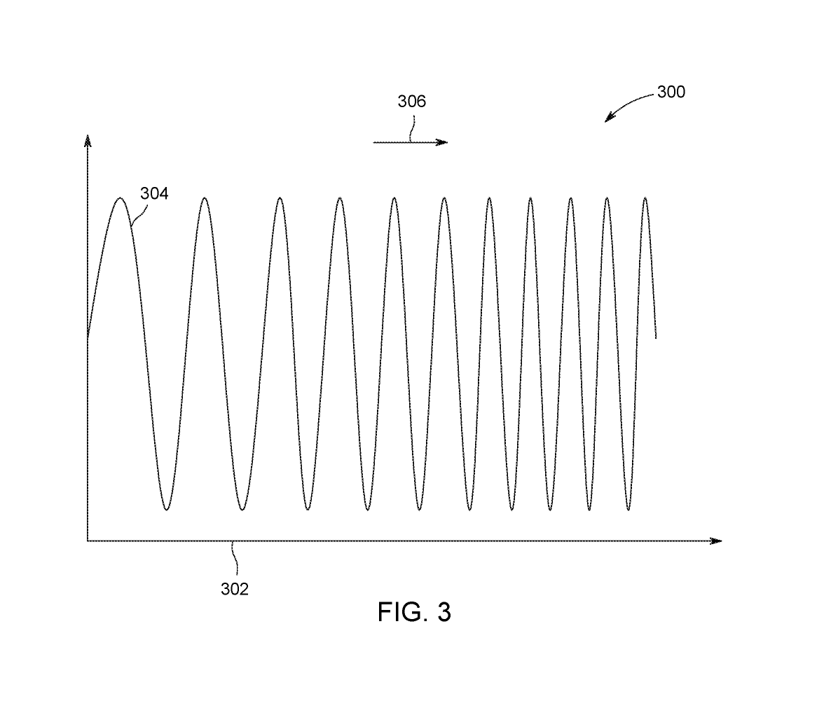

FIG. 3 is a graphical illustration 300 of a stimulation waveform 304 applied to the sensing material 114, 214 of a sensor 102, 250. The stimulation waveform 304 may be generated by the controller circuit 110, 210, 216. The stimulation waveform 304 may be an electrical stimulus having an amplitude (e.g., voltage, current, and/or the like) and a dynamic frequency. For example, the stimulation waveform 304 is shown plotted along a horizontal axis 302 representing time. Over time, the controller circuit 110, 210, 216 may adjust (e.g., increase, decrease) the frequency of the stimulation waveform 304. For example, as shown in FIG. 3, the controller circuit 110 may increase the frequency of the stimulation waveform 304 along the axis 302 in a direction of an arrow 306. In various embodiments, the stimulation waveform 304 may be a chirp and/or sweep signal.

Optionally, a range of the frequencies of the stimulation waveform 304 is adjusted by the controller circuit 110, 210, 216 based on a frequency bandwidth. The frequency bandwidth may be a defined range of frequencies centered at a resonance frequency of the sensor 102, 250 (e.g., configured to a part of a non-resonant or a resonant circuit). Additionally or alternatively, the range the frequency of the stimulation waveform 304 is adjusted by the controller circuit 110, 210, 216 based on one or more scanning bandwidths. The scanning bandwidths may be a range of frequencies that are non-resonant frequencies of the sensor 102, 250. For example, the scanning bandwidths may be utilized by the controller circuit 110, 210, 216 when the sensor 102, 250 is configured a part of a non-resonant circuit.

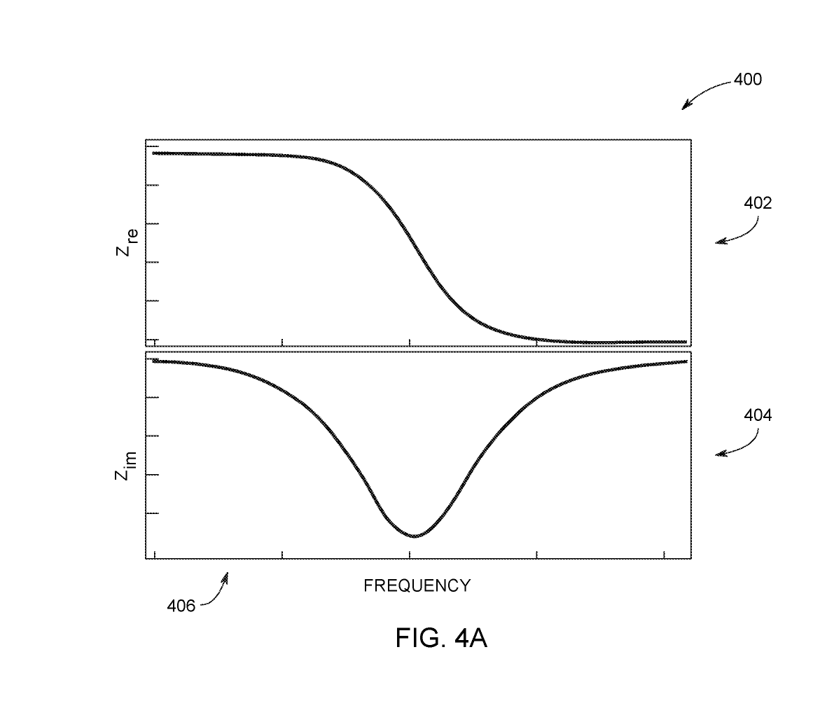

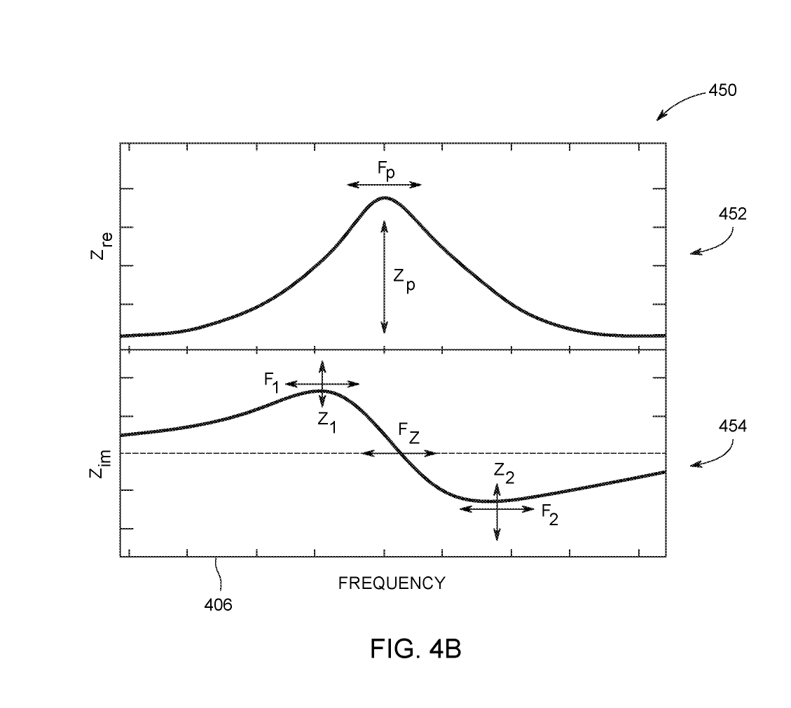

FIGS. 4A-4B show graphical illustrations measured response 400, 450 corresponding to an impedance response 402, 404, 452, 454 of the sensors 102 and 250, respectively, in accordance with an embodiment.

For example, the impedance responses 400, 450 may represent the impedance sensor response of the sensor 102, 250 (respectively) based on the stimulation waveform 304 generated by the controller circuit 110, 210, 216. The impedance responses 400, 450 includes several individually measured spectral parameters of the sensor 102, 250. The impedance responses 400, 450 are divided into real portions 402, 452 corresponding to the real impedance, Zre(f) of the impedance responses 400, 450, and imaginary portions 404, 454 of an imaginary impedance, Zim(f). The impedance responses 400, 450 are measured by the controller circuit 110, 210, 216 based on a measurement signal. For example, the controller circuit 110, 216 may receive the measurement signal from the electrodes (e.g., the electrodes 108-109, the antenna 218a-b) in contact with the sensing material 114, 214. The measurement signal is an electrical signal generated by the sensing material 114, 214 in response to the stimulation waveform 304 and the ambient environment. The measurement signal is representative of the impedance response of the sensing material 114. For example, the measurement signal may have electrical characteristics (e.g., voltage, current, frequency, and/or the like), which may be utilized by the controller circuit 110, 210, 216 to calculate the impedance responses 400, 450.

Based on the impedance responses 400, 450, the controller circuit 110, 216 may calculate spectral parameters associated with the measured Zre(f) and Zim(f). For example, the spectral parameters may include the peak frequency position Fp and peak magnitude Zp of the real portion 452, Zre(f). The spectral parameters may include the resonant F1 and anti-resonant F2 frequencies of the imaginary portion 454, Zim(f), the impedance magnitudes Z1 and Z2 at F1 and F2 frequencies, respectively, and the zero-reactance frequency Fz. Additionally or alternatively, the controller circuit 110, 216 may calculate a quality factor.

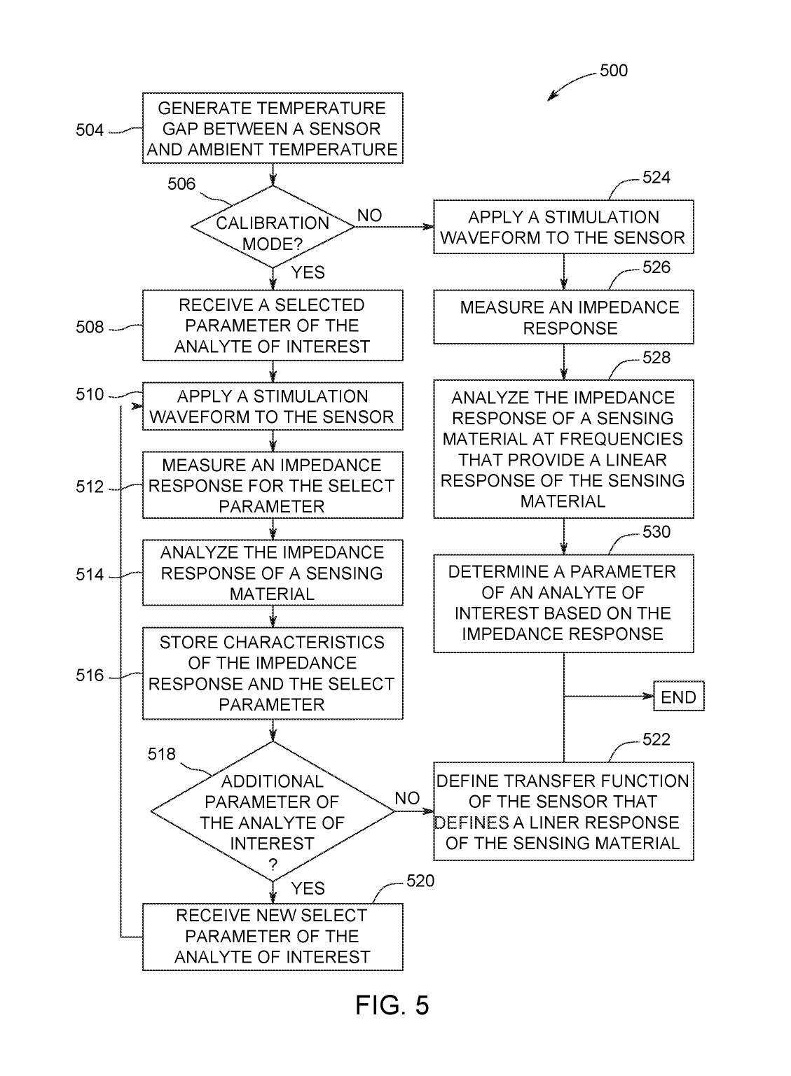

In connection with FIG. 5, from the calculated spectral parameters, resistance, capacitance, and/r the like of the sensing material 114, 214 may also be determined by a multivariate analysis. The multivariate analysis may be used to reduce the dimensionality of the impedance response, either from the real portion 402, 452 Zre(f), and imaginary portion 404, 454, Zim(f), of the impedance responses 400, 450 or from the calculated spectral parameters Fp, Zp, F1 and F2, and possibly other parameters to a single data point in a multidimensional space for selective quantization of the one or more analytes of interest.

FIG. 5 is a flow chart of a method 500 for detecting one or more analytes of interest, in accordance with an embodiment. The method 500, for example, may employ or be performed by structures or aspects of various embodiments (e.g., systems and/or methods) discussed herein. For example, the method 500 includes operations performed by and/or changes to the memory 104, the controller circuit 110, 210, 216, the sensor 102, 250, and/or the like. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. In various embodiments, portions, aspects, and/or variations of the method may be able to be used as one or more algorithms to direct hardware to perform one or more operations described herein.

It may be noted that although the method described below is in connection with the sensing system 100, the operations described may be utilized by the sensing system 200 (e.g., the controller circuit 210, 216, the sensor 250, the heater 222) shown in FIG. 2.

Beginning at 504, the heater 106 may generate a temperature gap (or difference) between the sensor 102 and ambient temperature. The temperature gap may represent a difference in temperatures between the sensor 102 and/or the components of the sensor 102 (e.g., at least one pair of electrodes 108-109, the sensing material 114) and the ambient temperature based on thermal energy generated by the heater 106. For example, the controller circuit 110 may instruct the heater 106 to generate thermal energy, which is received by the sensor 102. An amount of thermal energy generated by the heater 106 may be based on a temperature of the sensor 102 relative to the heater 106. For example, the controller circuit 110 may instruct the heater 106 to increase a temperature of the sensor 102 at least 50 degrees Celsius above the ambient temperature.

Additionally or alternatively, the temperature gap may be based on a predetermined temperature stored in the memory 104. For example, the controller circuit 110 may instruct the heater 106 to increase a temperature of the sensor 102 to at least 100 degrees Celsius. In another example, the controller circuit 110 may instruct the heater 106 to increase a temperature of the sensor 102 to at least 200 degrees Celsius or to at least 800 degrees Celsius.

At 506, the controller circuit 110 may determine if a user input is received indicative of a calibration mode. The calibration mode is utilized by the controller circuit 110 to define a transfer function for the sensor 102. The transfer function is utilized by the controller circuit 110 to determine a parameter (e.g., concentration) of the analyte of interest based on the impedance response, such as the measured spectral parameters of the impedance response. The controller circuit 110 receives the user input from the user interface 112. For example, a user of the sensing system 100 may utilize the user interface 112 to select the calibration mode, which generates a user input received by the controller circuit 110. Based on the user input, the controller circuit 110 may enter a calibration mode. Additionally or alternatively, the controller circuit 110 may automatically determine a calibration mode based on predetermined periodicity of the calibration mode. Additionally or alternatively, the controller circuit 110 may automatically determine a calibration mode based on a lack of transfer function stored in the memory 104.

If the sensing system 100 is in a calibration mode, at 508 the controller circuit 110 may receive a select parameter of the analyte of interest. The select parameter may correspond to a concentration and/or quantitative measure of an amount of the analyte of interest within the ambient environment of the sensor 102. For example, the controller circuit 110 may receive a user input from the user interface 112 indicative on the concentration of the analyte of interest. It may be noted that in various embodiments the select parameter may correspond to a temperature, pressure, conductivity, dielectric property, number of dielectric, metallic, chemical, or biological particles in the proximity or in contact with the sensor 102, dose of ionizing radiation, light intensity, and/or the like.

At 510, the controller circuit 110 may apply a stimulation waveform to the sensor 102. The stimulation waveform may be similar to and/or the same as the stimulation waveform 304 shown in FIG. 3. For example, the controller circuit 110 may generate the stimulation waveform 304 to the sensing material 114 utilizing the pair of electrodes 108-109 in contact with the sensing material 114. The stimulation waveform 304 is conducted through the electrodes 108-109 and received by the sensing material 114.

At 512, the controller circuit 110 may measure an impedance response for the select parameter. For example, the controller circuit 110 may receive a measurement signal generated by the sensing material 114 from the electrodes 108-109. The measurement signal is representative of an impedance response of the sensing material 114 in operational contact with the ambient environment. For example, the measurement signal may have electrical characteristics (e.g., voltage, current, frequency, and/or the like), which is utilized by the controller circuit 110 to calculate the impedance response. Optionally, the impedance response may be similar to and/or the same as the impedance response 400 shown in FIG. 4.

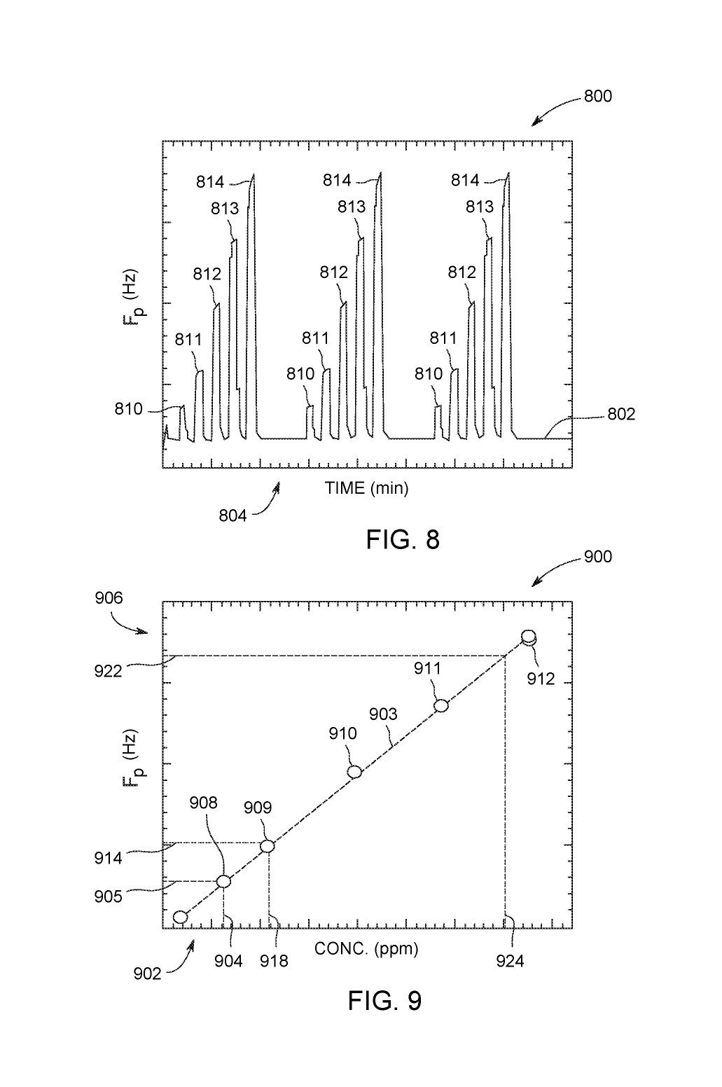

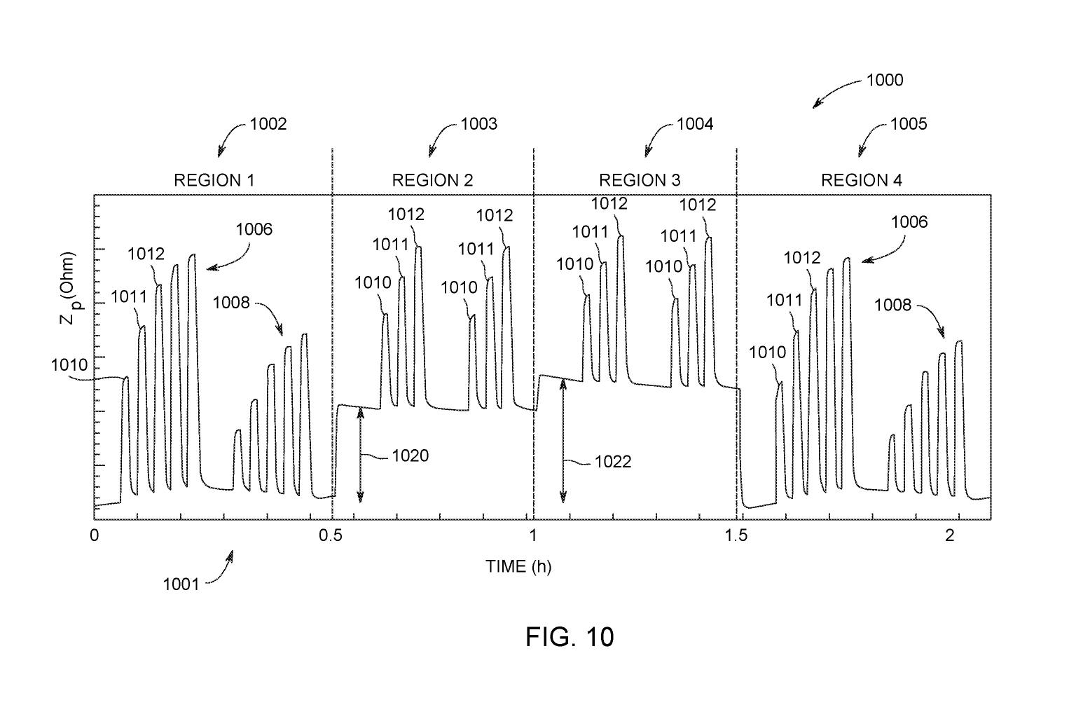

At 514, the controller circuit 110 may analyze the impedance response of the sensing material 114. For example, the controller circuit 110 may calculate one or more spectral parameters based on a real portion (e.g., Fp, Zp) and/or imaginary portion (e.g., F1, F2, Fz, Z1, Z2) of the impedance response. The controller circuit 110 may be configured to analyze the spectral parameters that provide a linear response (e.g., as shown in FIGS. 8-9) of the sensing material 114 to the analyte of interest and reject effects of interference analytes (e.g., analytes that are not the analyte of interest).

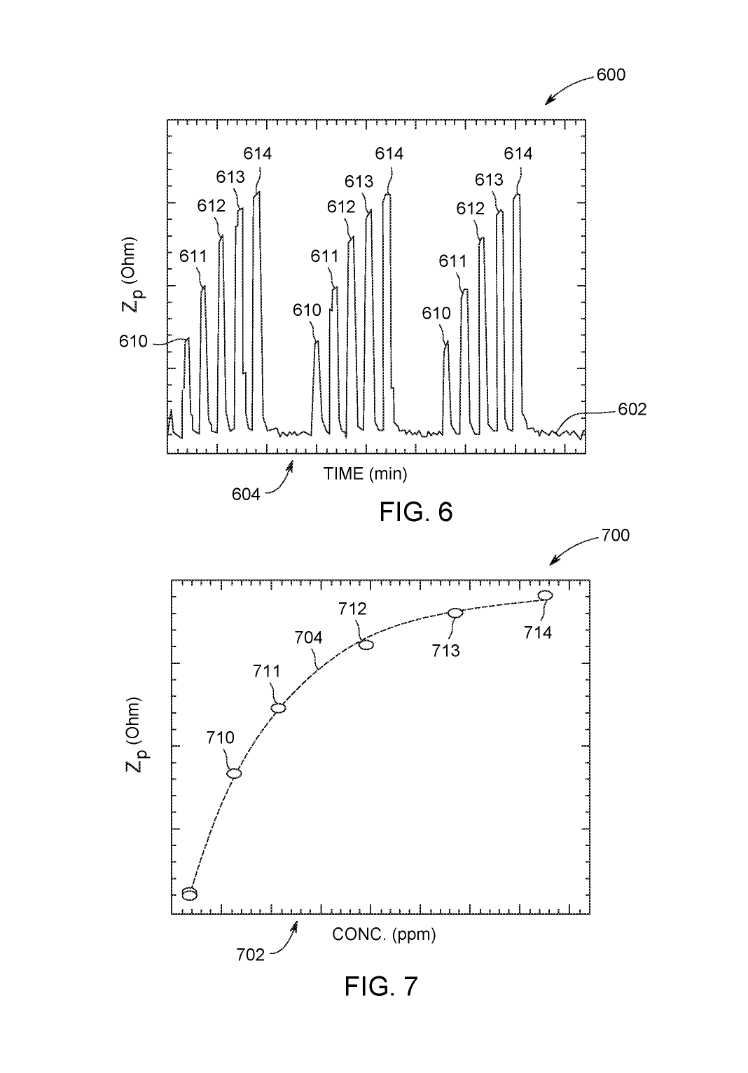

As a non-limiting example, in connection with FIGS. 6-7, a conventional resistance sensor is connected to a resonant circuit. This conventional resistance sensor is based on a SnO2 metal oxide and may detect an analyte of interest (e.g., methane gas, hydrogen, isobutane, ethanol).

For conventional sensor operation, the conventional sensor was heated to its prescribed working temperature of 300 degrees Celsius. Measurements of resonant spectra were done using an impedance analyzer. Zp response of the resonant sensor circuit is directly proportional to the resistance of this conventional resistance sensor.

FIG. 6 is a graphical illustration 600 of a spectral parameter 602 calculated from a conventional sensor. The spectral parameter 602 is a peak magnitude Zp plotted along a horizontal axis 604 representing time.

The conventional sensor was exposed to different concentrations (e.g., 111 ppm, 222 ppm, 444 ppm, 667 ppm, 889 ppm) of the analyte of interest (e.g., methane gas) and a dry air in between the exposures over time.

The spectral parameter 602 response based on the exposure to the different concentrations of the analyte of interest is represented by a non-linearity of the peaks 610-614 of the spectral parameter 602. Each of the peaks 610-614 may have an amplitude based on the concentrations of the analyte of interest exposed to the conventional sensor. For example, the amplitude of the peak 610 is less than the amplitude of the peak 613 representing the concentration of the analyte of interest of the peak 610 is less than at the peak 613. In connection with FIG. 7, a calibration curve 704 may be defined based on the peaks 610-614.

FIG. 7 is a graphical illustration 700 of the concentration curve 704 of the conventional sensor based on the spectral parameter 602 response. The concentration curve 704 is constructed from the spectral parameter 602, such as using the peaks 610-614. For example, the concentration curve 704 is constructed from using data points 710-714 based on the amplitudes of the peaks 610-614. It may be noted that the concentration curve 704 is non-linear (e.g., power law).

Representing embodiments described herein, in connection with FIGS. 8-9, the spectral parameter 802 of the impedance response of the sensor 102 is analyzed by the controller circuit 110 having a linear response.

FIG. 8 is a graphical illustration 800 of the spectral parameter 802 calculated by the controller circuit 110 of the sensor 102 configured as a resonant sensor and/or the sensor 250. The spectral parameter 802 is a peak frequency Fp plotted along a horizontal axis 804 representing time. The sensor 102, operating in the resonant mode, was exposed to different concentrations (e.g., 111 ppm, 222 ppm, 444 ppm, 667 ppm, 889 ppm) of the analyte of interest and a dry air in between the exposures over time.

The spectral parameter 802 response based on the exposure to the different concentrations of the analyte of interest (e.g., methane gas) are represented by a linearity of peaks 810-814 of the spectral parameter 802. Each of the peaks 810-814 may have an amplitude based on the concentration of the analyte of interest presented to the sensor 102. For example, the amplitude of the peak 810 is less than the amplitude of the peak 813 representing the concentration of the analyte of interest of the peak 810 is less than at the peak 813. In connection with FIG. 9, a calibration curve 903 may be defined based on the peaks 810-814.

FIG. 9 is a graphical illustration 900 of the concentration curve 903 of the sensor 102 based on the spectral parameter 802 response. The concentration curve 903 is constructed from the spectral parameter 802, such as the peaks 810-814. For example, the concentration curve 903 is constructed from using data points 908-912 based on the amplitudes of the peaks 810-814. It may be noted that the concentration curve 903 is linear (e.g., not power law). This unexpected discovery shows that the sensor 102 produces a highly linear response upon exposure to the different concentrations of an analyte of interest (e.g., methane gas) of the spectral parameter 802. Additionally or alternatively, the concentration curve 903 is further shown having a monotonic response.

The graphical illustration 900 represents the linear relationship of characteristics of an impedance response of the sensor 102 and parameters of the analyte of interest, in accordance with an embodiment. The characteristics of the impedance response may correspond to the frequencies of the real portion of the impedance response, which is plotted along a vertical axis 906. The parameters of the analyte of interest may correspond to the concentration of the analyte of interest (e.g., parts per million (ppm)) in the ambient environment of the sensor 102. The graphical illustration 900 includes the plurality of data points 908-912. Each of the data points 908-912 may correspond to frequencies of the real portion of the impedance responses at different concentrations of the analyte of interest. For example, data point 908 may correspond to a concentration at 904 with the frequency at 905 of the real portion of the impedance response. In another example, the data point 909 may correspond to a concentration at 918 with the frequency at 914 of the real portion of the impedance response.

The data points 908-912 define a linear response of the concentration curve 903 of the frequencies of the real portion of the impedance response of the sensor 102 at different concentrations. Based on the linear response of the concentration curve 903, the controller circuit 110 may define a transfer function of the sensor 102. The transfer function may be utilized by the controller circuit 110 to determine a characteristic of the analyte of interest based on one or more spectral parameters calculated from the impedance response (e.g., at 530 in FIG. 5).

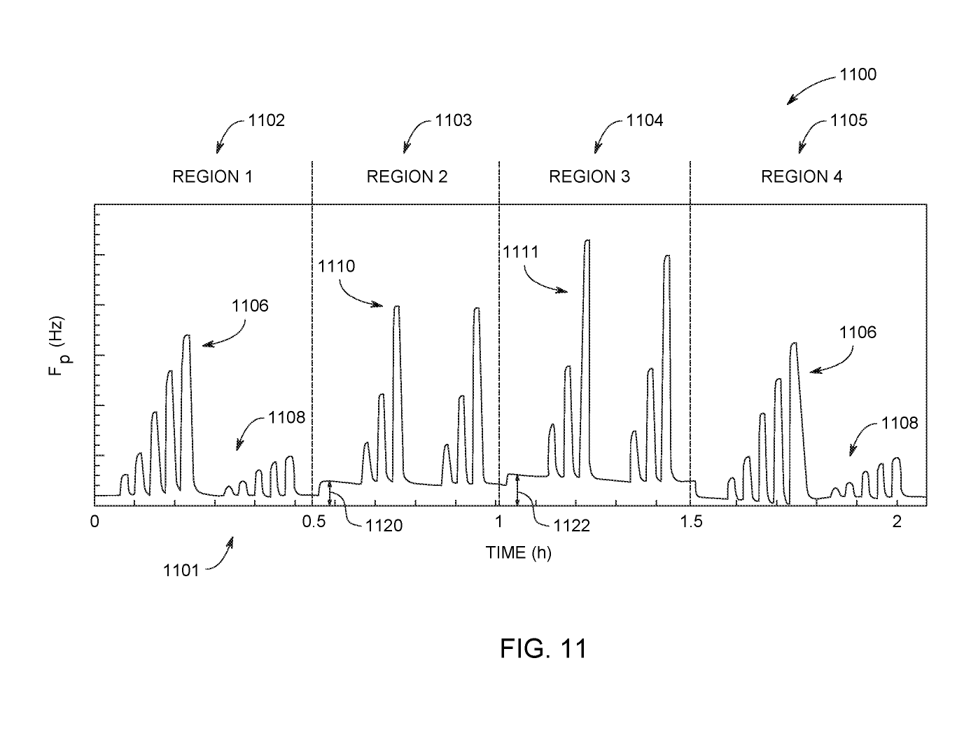

In connection with FIGS. 10 and 11, the sensing material 114 is configured to have the impedance response that provides a reduction of effect of interferences relative to the resistance response of a conventional sensor.

FIG. 10 is graphical illustration 1000 of a typical effect of an analyte of interest (e.g., methane gas) and ambient humidity on a spectral parameter of a conventional sensor. The spectral parameter shown in FIG. 10 is a peak magnitude Zp plotted along a horizontal axis 1001 representing time. The conventional sensor was exposed individually to the analyte of interest and water vapor as separate exposures and to the mixtures of the analyte of interest and water vapor. The graphical illustration 1000 includes four experimental regions 1002-1005 of gas exposures.

At the region 1002 and 1005, the conventional sensor was exposed to five concentrations of the analyte of interest (e.g., 111, 222, 444, 667, 889 ppm) and dry air in between the analyte of interest exposures to form a series of peaks 1006 of the spectral parameter. The series of peaks 1006 include peaks 1010-1012 based on concentrations at 111, 222, and 444 ppm of the analyte of interest. Subsequent to the concentrations of the analyte of interest, water vapor concentrations such as having different ambient humidity (e.g., at 9, 18, 36, 53, and 71 percent) is exposed to the conventional sensor and dry air in between the humidity exposures to form a series of peaks 1008.

At the region 1003, the conventional sensor was periodically exposed to three concentrations of the analyte of interest (e.g., 111, 222, 444 ppm) concurrently with a water vapor of 18 percent relative humidity to form peaks 1010-1012 of the spectral parameter.

At the region 1004, the conventional sensor was periodically exposed to three concentrations of the analyte of interest (e.g., 111, 222, 444 ppm) concurrently with a water vapor of 36 percent relative humidity to form the peaks 1010-1012 of the spectral parameter.

It may be noted that FIG. 10 illustrates the conventional sensor is significantly affected by the ambient humidity exposed to conventional sensor, which shifts an amplitude of the peaks 1010-1012 of the spectral parameter. For example, the concentrations of the analyte of interest for the peaks 1010-1012 are the same for the regions 1002-1005. However, due to the ambient humidity of the regions 1003-1004, the amplitudes of the peaks 1010-1012 are shifted by shift magnitudes of 1020 and 1022, respectively, due to the ambient humidity relative to the amplitude of the peaks 1010-1012 shown in regions 1002 and 1005.

FIG. 11 is graphical illustration 1100 of a typical effect of an analyte of interest (e.g., methane gas) and ambient humidity on a spectral parameter of the sensor 102. The spectral parameter shown in FIG. 11 is a peak frequency Fp plotted along a horizontal axis 1101 representing time. The sensor 102 was exposed individually to the analyte of interest and water vapor as separate exposures and to the mixtures of the analyte of interest and water vapor. The graphical illustration 1100 includes four experimental regions 1102-1105 of gas exposures. Unexpectedly, we have found that when Fp measurements were performed with respect to the sensor 102, a significantly reduced effect of water vapor (e.g., ambient humidity) was observed as shown in FIG. 11. Thereby, the new disclosed principle of analyte of interest utilizing the sensor 102, provides a significantly reduced effects of humidity.

For example, at the region 1102 and 1105, the conventional sensor was exposed to five concentrations of the analyte of interest (e.g., 111, 222, 444, 667, 889 ppm) and dry air in between the analyte of interest exposures to form a series of peaks 1006 of the spectral parameter. Subsequent to the concentrations of the analyte of interest, water vapor concentrations such as having different ambient humidity (e.g., at 9, 18, 36, 53, and 71 percent) are presented to the conventional sensor and dry air in between the humidity exposures to form a series of peaks 1108. At the region 1103, the conventional sensor was periodically exposed to three analyte of interest concentrations (e.g., 111, 222, 444 ppm) concurrently with a water vapor of 18 percent relative humidity to form a series of peaks 1110 of the spectral parameter. At the region 1104, the conventional sensor was periodically exposed to three analyte of interest concentrations (e.g., 111, 222, 444 ppm) concurrently with a water vapor of 36 percent relative humidity to form the peaks 1111 of the spectral parameter.

At regions 1103 and 1104, the series of peaks 1110 and 1111 of the spectral parameter is affected by the ambient humidity exposed to the sensor 102, which shifts an amplitude of the peaks 1110 and 1111 relative to the series of peaks 1106 by shift magnitudes 1120 and 1122, respectively. It may be noted, that the shift magnitudes 1120 and 1120 of the regions 1103 and 1104 are significantly less than the shift magnitudes 1020 and 1022 shown in regions 1002 and 1005. For example, the sensing material 114 and/or sensor 102 is configured to reduce effects of humidity of the impedance response by ten times relative to the conventional sensor shown in FIG. 10. Additionally or alternatively, the sensing material 114 and/or sensor 102 may be configured to reduce effects of humidity of the impedance response to approximately zero relative to the conventional sensor shown in FIG. 10.

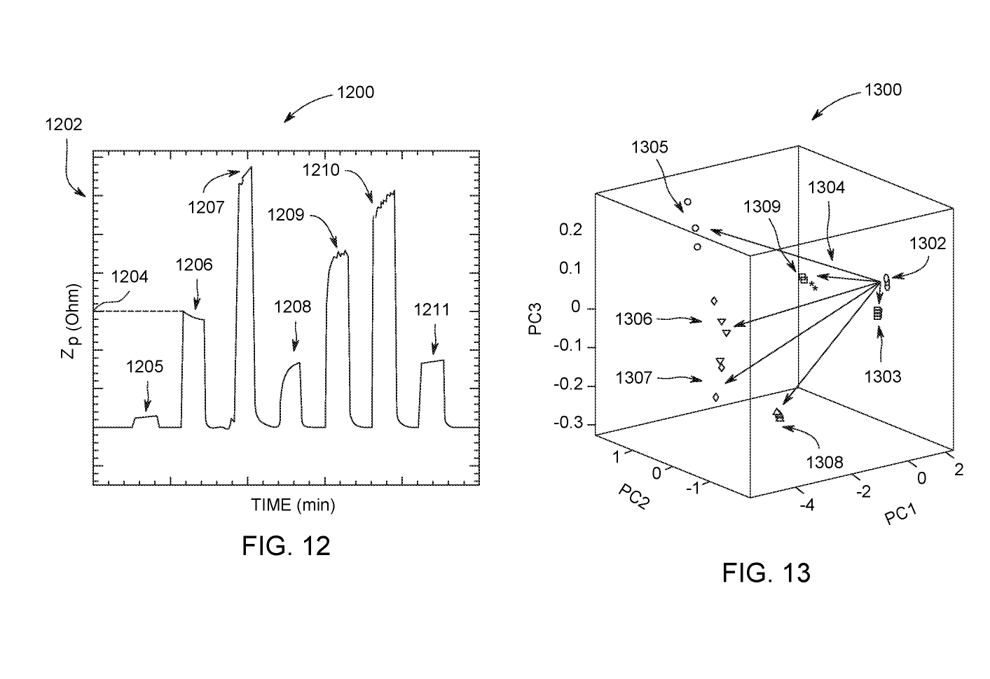

In connection with FIG. 12, the controller circuit 110 may analyze the peak height Zp of the real portion of the resonant impedance response that includes multiple analytes (e.g., water, methane, tetrahydrofuran, benzene, ethyl acetate, ethanol, toluene, and/or the like). For example, a spectral parameter 1200 may be in response to the sensor 102 being exposed individually to different analytes (e.g., vapors) as separate exposures with dry air interposed between the exposures of each analyte. It may be noted that the controller circuit 110 may analyze additional spectral parameters concurrently and/or simultaneously with the each other. For example, the controller circuit 110 may analyze the frequencies of the real portion of the impedance response concurrently and/or simultaneously with the impedance magnitudes of the real portion of the impedance response.

FIG. 12 is a graphical illustration of a spectral parameter 1200 calculated from an impedance response of the sensor 102, in accordance with an embodiment. The spectral parameter 1200 may correspond to impedance magnitude Zp calculated from a real portion of the resonant impedance response. The magnitudes of the impedance Zp are plotted along a vertical axis 1202. The spectral parameter 1200 shown in FIG. 12 shows the sensor 102 has a cross-sensitivity to different analytes. For example, the spectral parameter based on the ambient environment in contact with the sensing material 114, includes multiple response peaks 1205-1211. Each of the peaks 1205-1211 may correspond to a different analytes (e.g., gas or vapor) detected within the ambient environment of the sensor 102. For example, one of the peaks 1205-1211 may correspond to water, methane, tetrahydrofuran, benzene, ethyl acetate, ethanol, toluene, and/or the like.

As depicted in FIG. 12, responses Zp to different gases or vapors have different magnitudes. The controller circuit 110 may compare the magnitudes of the Zp response to an analyte parameter database to determine which of the frequency peaks correspond to the analyte of interest. The analyte parameter database may be stored in the memory 104. The analyte parameter database may include a plurality of analytes each having corresponding spectral parameters. For example, the analyte parameter database may include a plurality of analytes with corresponding real frequencies. The controller circuit 110 may identify the analyte of interest within the analyte parameter database with corresponding real frequencies that include the frequency at 1204. The controller circuit 110 may determine that the frequency peak 1206 that includes the frequency at 1204 corresponds to the analyte of interest, and filter and/or reject the responses 1205, 1207-1211 corresponding to interference and/or analytes not of interest.

Additionally or alternatively, in connection with FIG. 13, the controller circuit 110 may execute a multivariate analysis of the impedance response of the sensor 102 to multiple analytes performed using spectral parameters Fp, Zp, F1, F2, Z1, and Z2 and processing these outputs using a principal components analysis (PCA). Based on the PCA, the controller circuit 110 may eliminate the effects of volatiles (e.g., analytes not the analyte of interest, intereference) and provide an accurate response and/or to isolate the analyte of interest into its unique response direction.

FIG. 13 is a graphical illustration 1300 of one embodiment of a principal components analysis of a plurality of spectral parameters. For example, the graphical illustration 1300 is calculated by the controller circuit 110 by executing a PCA analysis of spectral parameters Fp, Zp, F1, F2, Z1, and Z2 calculated from an impedance response of the sensor 102. Based on the multiple outputs 1302-1309 of the PCA response, the controller circuit 110 may discriminate between different analytes utilizing its unique response direction. Each of the multiple outputs 1302-1309 correspond to a different analyte. For example, the output 1302 may represent dry air (e.g., control having no analytes), the output 1303 may represent water, the output 1304 may represent benzene, the output 1305 may represent ethyl acetate, the output 1306 represent tetrahydrofuran, the output 1307 may represent ethanol, the output 1308 may represent methane, and the output 1309 may represent toluene.

Returning to FIG. 5, at 516, the controller circuit 110 may store characteristics of the impedance response and the corresponding select parameter in the memory 104. The characteristics of the impedance response may correspond to the spectral parameters calculated at 514. For example, the controller circuit 110 may store the response magnitude Zp of sensor resistance at 1204 (FIG. 12) based on the resistance magnitude peak 1206 corresponding to the analyte of interest and the corresponding select parameter in the memory 104. The controller circuit 110 may link the magnitude at 1204 to the select parameter, such as concentration, of the analyte of interest in the memory 104. Optionally, the characteristic and the select parameter may be a data point (e.g., such as the data points 908-912 shown in FIG. 9) utilized to define a transfer function of the sensor 102. Additionally or alternatively, the select parameter may correspond to a response direction of the analyte of interest. For example, the controller circuit 110 may store a direction of the output 1308 in the memory 104 corresponding to the analyte of interest.

At 518, the controller circuit 110 may determine whether additional parameters of the analyte of interest are available. For example, the controller circuit 110 may receive a user input from the user interface 116 indicative of additional parameter of the analyte of interest is available. In another example, the controller circuit 110 may have a predetermined threshold of parameters of the analyte of interest, and may determine that additional parameters are available until the predetermined threshold has been reached.

If additional parameters of the analyte of interest are available, at 520 the controller circuit 110 receives a new select parameter of the analyte of interest. For example, the controller circuit 110 may receive a user input from the user interface 112 indicative on the new select parameter (e.g., a new concentration) of the analyte of interest.

If there are no additional parameters of the analyte of interest, at 522 the controller circuit 110 may define a transfer function of the sensor 102 that defines a linear response of the sensing material based on the analyte of interest. For example, in connection with FIG. 9, based on the concentration curve 903 having a linear response, the controller circuit 110 may define a transfer function of the sensor 102. The transfer function is utilized by the controller circuit 110 to determine a characteristic of the analyte of interest based on one or more spectral parameters calculated from the impedance response.

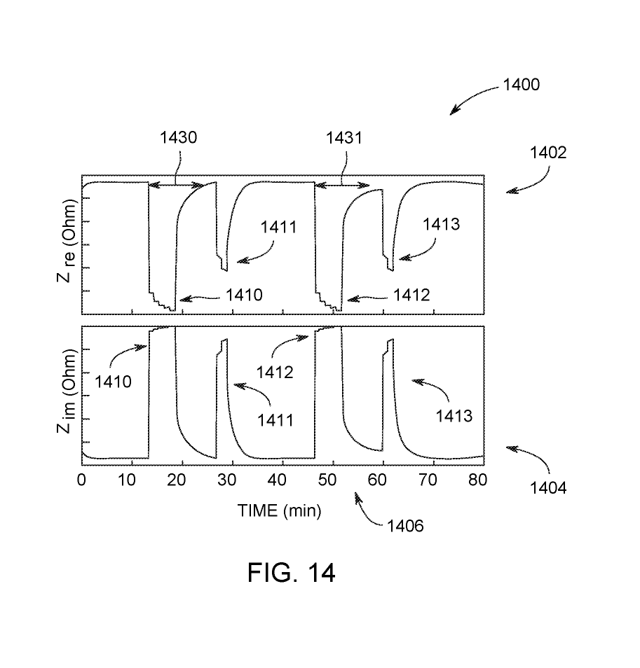

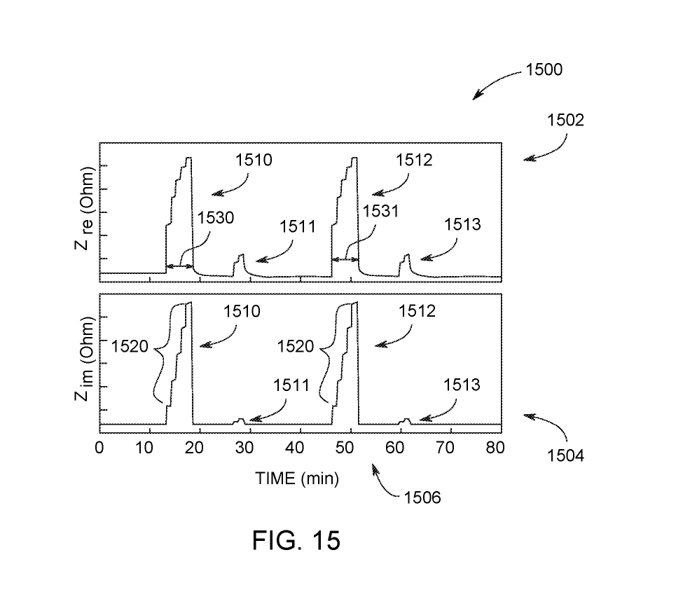

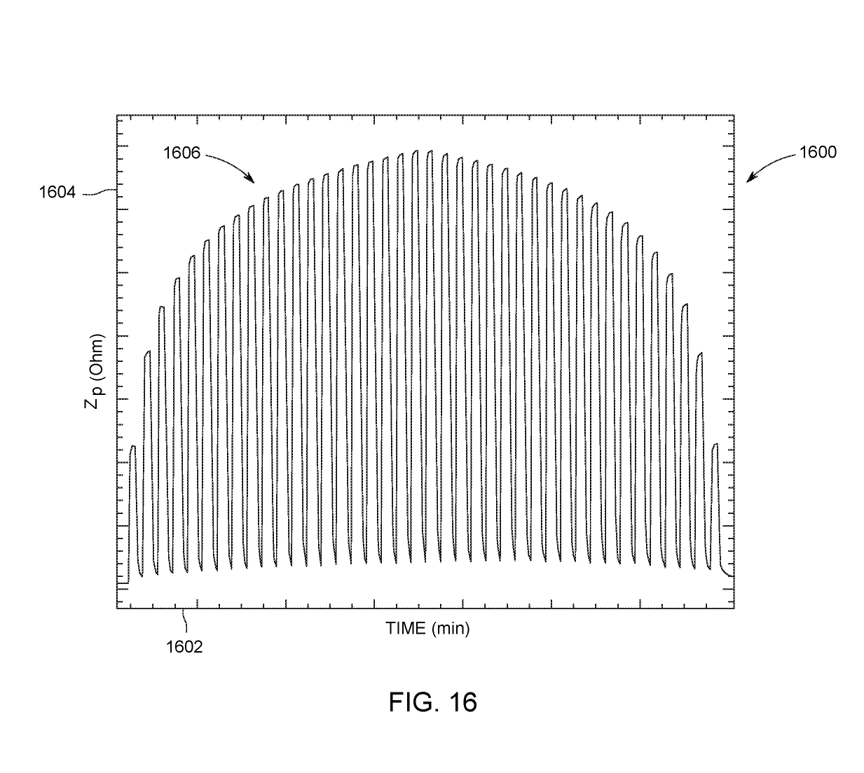

Additionally or alternatively, in connection with FIGS. 14-15, the sensor 102 may be configured to perform in a non-resonance impedance mode. For example, the controller circuit 110 may increase a temperature of the sensor 102, utilizing the heater 106 to a temperature of 300 degrees Celsius. The sensor 102 may receive a stimulation waveform from the controller circuit 110 having a frequency that is not at a resonance frequency of the sensor 102. While receiving the stimulation waveform, the sensor 102 may be exposed individually to different analytes (e.g., methane, water vapor) at increasing concentrations at separate exposures interposed by dry air in between exposures of the analytes to form separate peaks. For example a first analyte (e.g., methane) concentrations were at 555, 1111, 1667, 2222, and 2778 ppm. A second analyte, such as water vapor, concentrations generated were 27 and 53 percent relative humidity.

FIG. 14 are graphical illustrations 1400 of spectral parameters 1402, 1404 of one embodiment of a measured response of the sensor 102. For example, the controller circuit 110 may generate a stimulation waveform received by the sensor 102 having a frequency at 0.1 kHz. The sensor 102 may generate a measurement signal, which is received and measured by the controller circuit 110 representative of the impedance response of the sensor 102. The controller circuit may calculate the spectral parameters 1402, 1404 based on the impedance response over time, the horizontal axis 1406. For example, the spectral parameter 1402 may represent a real impedance Zre, and the spectral parameter 1404 may represent an imaginary impedance Zim of the impedance response. Each of the spectral parameters 1402, 1404 may include peaks 1410-1413 representing the analytes exposed by the sensor 102. For example the peaks 1410 and 1412 may represent the exposure of the first analyte (e.g., methane), and the peaks 1411 and 1413 may represent the exposure of the second analyte, such as water vapor.

FIG. 15 are graphical illustrations 1500 of spectral parameters 1502, 1504 of one embodiment of a measured response of the sensor 102. For example, the controller circuit 110 may generate a stimulation waveform received by the sensor 102 having a frequency at 100 kHz. The sensor 102 may generate a measurement signal, which is received and measured by the controller circuit 110 representative of the impedance response of the sensor 102. The controller circuit may calculate the spectral parameters 1502, 1504 based on the impedance response over time, the horizontal axis 1506. For example, the spectral parameter 1502 may represent a real impedance Zre, and the spectral parameter 1504 may represent an imaginary impedance Zim of the impedance response. Each of the spectral parameters 1502, 1504 may include peaks 1510-1513 representing the analytes exposed by the sensor 102. For example, the peaks 1510 and 1512 may represent the exposure to the first analyte, and the peaks 1511 and 1513 may represent the exposure to the second analyte.

It may be noted that the sensing material 114 is configured such that the stimulation waveform and/or operation of the sensor 102 at high frequencies (e.g., at and/or above 100 kHz), as shown in FIG. 15, provides an improved response linearity to an analyte of interest (e.g., methane) relative to lower frequencies, as shown in FIG. 14. For example, the peaks 1510 and 1512 include a defined linear response 1520 based on the increase in concentration of the first analyte exposed to the sensor 102 over time during the peaks 1510 and 1512.

Additionally or alternatively the sensing material 114 is configured to have the impedance response that provides a reduction of effects of interferences over the resistance response of the sensing material 114. For example, the operation of the sensor 102 at high frequencies (e.g., at and/or above 100 kHz) provides suppression of the impedance response to an interference by water vapor (e.g., humidity) exposed by the sensing material 114. In connection with FIGS. 14-15, the peaks 1411, 1413, 1511, and 1513 correspond to the exposure of the sensor 102 to the second analyte representing water vapor. Based on the difference in operational frequency of the sensor 102, the peaks 1511 and 1513 have a lower amplitude than the peaks 1411 and 1413. It may be noted that the peaks 1511 and 1513 of the spectral parameter 1504 representing the imaginary part of impedance Zim provides a stronger suppression of response to the second analyte compared to the peaks 511 and 1513 of the spectral parameter 1502 representing the real part of impedance Zre. For example, the sensing material 114 and/or sensor 102 is configured to reduce effects of humidity of the impedance response by ten times relative to the conventional sensor shown in FIG. 14. Additionally or alternatively, the sensing material 114 and/or sensor 102 may be configured to reduce effects of humidity of the impedance response to approximately zero relative to the conventional sensor shown in FIG. 14.

Additionally or alternatively, the sensing material 114 is configured to have the impedance response that provides a reduction of recovery time based on a frequency of the stimulation waveform. For example, the peaks 1410 and 1412 have a corresponding peak width 1430 and 1431, respectively. During operation of the sensor 102 at high frequencies (e.g., at and/or above 100 kHz), the peak width decreases relative to operation at lower frequencies (e.g., that formed the peak widths 1430 and 1431). For example, the peak widths 1530 and 1531 of the peaks 1510 and 1512, respectively, have a shorter length relative to the peaks widths 1430 and 1431 representing a reduced recovery time.

Additionally or alternatively, the sensing material 114 is configured to have the impedance response that provides improvement of the baseline stability over the resistance response of a conventional sensor.

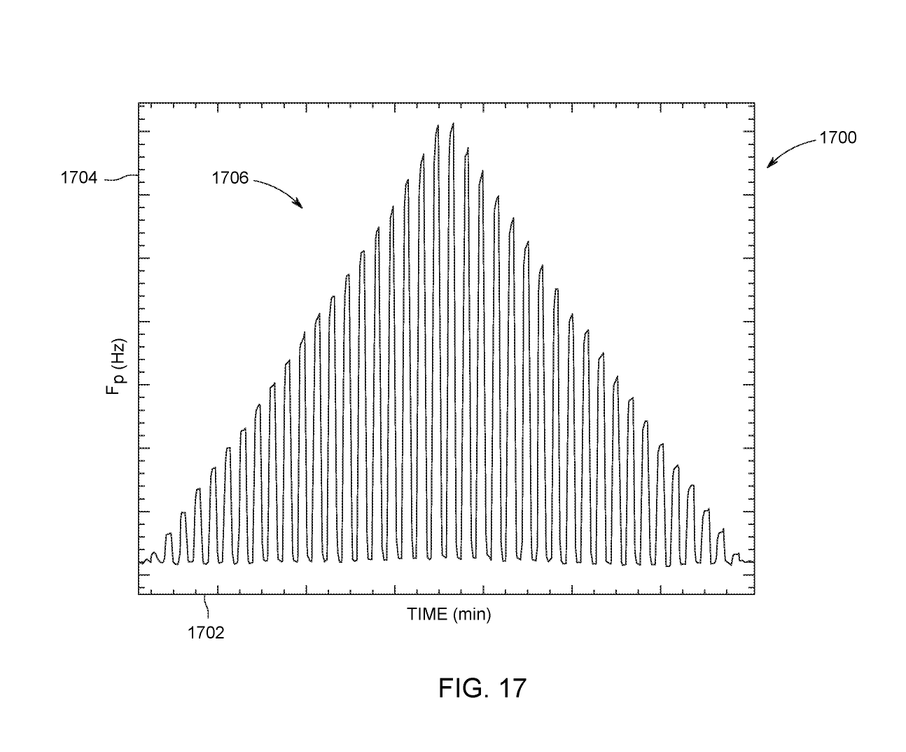

FIG. 16 is a graphical illustration 1600 of a spectral parameter 1606 of an embodiment calculated by the controller circuit 110 of a conventional sensor configured as a resonant sensor. The spectral parameter 1606 includes a peak magnitude Zp (along a vertical axis 1604) plotted along a horizontal axis 1602 representing time. The conventional sensor operating in the resonant mode was exposed to different concentrations of the analyte of interest (e.g. methane) and a dry air in between the exposures over time. The different concentrations of the analyte of interest were presented to the sensor in the order of increasing concentrations (e.g., 0, 44.4, 88.9, 133, 178, 222, 267, 311, 356, 400, 444, 489, 533, 578, 622, 667, 711, 756, 800, 844 and 889 ppm) followed by the order of decreasing concentrations (e.g., 889, 844, 800, 756, 711, 667, 622, 578, 533, 489, 444, 400, 356, 311, 267, 222, 178, 133, 88.9, 44.4 and 0 ppm). Such presentation of the analyte concentrations provided the ability to access the sensor linearity upon increasing and decreasing analyte concentrations. As depicted in FIG. 16, the sensor had a non-linear response (e.g. power law) as a function of analyte concentrations.

FIG. 17 is a graphical illustration 1700 of the spectral parameter 1706 calculated by the controller circuit 110 of the sensor 1102 configured as a resonant sensor. The spectral parameter 1706 is a frequency peak position Fp (along a vertical axis 1704) plotted along a horizontal axis 1702 representing time. The conventional sensor 102 operating in the resonant mode was exposed to different concentrations of the analyte of interest (e.g. methane) and a dry air in between the exposures over time. The different concentrations of the analyte of interest were presented to the sensor in the order of increasing concentrations (e.g. 0, 44.4, 88.9, 133, 178, 222, 267, 311, 356, 400, 444, 489, 533, 578, 622, 667, 711, 756, 800, 844, and 889 ppm) followed by the order of decreasing concentrations (e.g. 889, 844, 800, 756, 711, 667, 622, 578, 533, 489, 444, 400, 356, 311, 267, 222, 178, 133, 88.9, 44.4, and 0 ppm). Such presentation of the analyte concentrations provided the ability to access the sensor linearity upon increasing and decreasing analyte concentrations. As depicted in FIG. 17, the sensor had a linear response as a function of analyte concentrations.

It may be noted that other types of metal oxide sensors may be utilized to benefit from the subject matter described herein of non-resonant and resonant impedance measurements to obtain improved response linearity to an analyte of interest, suppression of response to humidity and other interferences, rapid recovery time, improved baseline stability and the ability to discriminate between different analytes by bringing the response to gas of interest was into its unique response direction.

Additionally or alternatively, the sensing system 100, 200 may include additional sensors, such as a humidity sensor (not shown), a temperature sensor (not shown), and or the like. The controller circuit 110 may adjust the impedance response based on the measurements of the additional sensors to define the transfer function. Additionally or alternatively, the controller circuit 110 may include measurements of the humidity sensor and/or the temperature sensor to define the transfer function.

Returning to FIG. 5, if the sensing system 100 is not in a calibration mode, at 524 the controller circuit 110 may apply a stimulation wave form to the sensor 102. The stimulation waveform may be similar to and/or the same as the stimulation waveform 304 shown in FIG. 3. For example, the controller circuit 110 may generate the stimulation waveform 304 to the sensing material 114 utilizing the pair of electrodes 108-109 in contact with the sensing material 114. The stimulation waveform 304 is conducted through the electrodes 108-109 and received by the sensing material 114.

At 526, the controller circuit 110 may measure an impedance response. For example, the controller circuit 110 may receive a measurement signal generated by the sensing material 114 from the electrodes 108-109. The measurement signal is representative of an impedance response of the sensing material in operational contact with the ambient environment. For example, the measurement signal may have electrical characteristics (e.g., voltage, current, frequency, and/or the like), which is utilized by the controller circuit 110 to calculate the impedance response. Optionally, the impedance response may be similar to and/or the same as the impedance response 400 shown in FIG. 4.

At 528, the controller circuit 110 may analyze the impedance response of the sensing material 114 at frequencies that provide a linear response of the sensing material 114. The controller circuit 110 may calculate one or more spectral parameters based on a real portion (e.g., Fp, Zp) and/or imaginary portion (e.g., F1, F2, Fz, Z1, Z2) of the impedance response. Optionally, the one or more spectral parameters calculated by the controller circuit 110 may be based on a transfer function defining the linear relationship between the impedance response and a parameter of the analyte of interest.

For example, in connection with FIG. 9, the transfer function of the sensor 102 may be based on a peak frequency (Fp) of the real portion of the impedance response, along the vertical axis 906 and a concentration of the analyte of interest, along the horizontal axis 902. The controller circuit 110 may select frequencies of the real portion of the impedance response to reject and/or filter out effects of interferences (e.g., from analytes not of interest) based on the analyte parameter database stored in the memory 104 as described above. For example, the controller circuit 110 may determine the peak frequency of the impedance response corresponding to the analyte of interest is at 922.

At 530, the controller circuit 110 may determine a parameter of an analyte of interest based on the impedance response. For example, the controller circuit 110 may utilize the transfer function stored in the memory 104 to determine a concentration of the analyte of interest within the ambient environment of the sensor 102.

Additionally alternatively, based on the parameter of the analyte of interest, the controller circuit 110 may automatically perform one or more responsive actions. Optionally, the one or more responsive action may be configured to alert a user and/or remote system. For example, if the parameter (e.g., concentration) is above a predetermined threshold the controller circuit 110 may display and/or initiate an auditory alert on the user interface 112.

The sensors 102, 250 described herein are applicable for diverse applications. In one non-limiting example, the sensor 102, 250 may be positioned and/or installed on an unmanned or manned vehicle. For example, the vehicle may be an aerial vehicle (e.g., drone, airplane, helicopter, and/or the like), automobile (e.g., car, truck, van, and/or the like). The vehicle may be positioned and/or traverse to one or more remote sites, and configured to collect ambient air pollution data of the one or more remote sites. For example, the controller circuit 102, 210 may be configured to analyze an impedance response of the sensor 102, 250 to one or more analytes of interest that represent air pollution (e.g., sulfur oxide, nitrogen oxide, carbon monoxide, methane, ammonia, and/or the like). Based on the concentration levels of the one or more analytes of interest representing air pollution, the control circuit 102, 210 may determine the ambient air pollution of proximate to the vehicle within the remote site. Optionally, the vehicle may include an RF circuit configured to wirelessly transmit the air pollution data (e.g., concentration information of the one or more analytes of interest) to a remote system (e.g., server, air pollution monitoring system).

In another non-limiting example, the sensor 102, 250 may be installed to monitor natural gas transmission infrastructure. A particularly urgent problem with cities is the leakage of methane gas into the ambient environment. There are currently thousands of miles natural gas pipes under the streets of major US cities in the United States alone. Many of these cities have old natural gas piping that have been subjected to massive wear and tear, particularly at cities where old infrastructure exists. As a consequence, methane gas leaks have unfortunately become quite common at these cities. A system (e.g., the sensing system 100, the sensing system 200) configured for collecting ambient methane emission data from city streets that includes the 102, 250 sensing system and the sensor material 114, 214 located on existing urban infrastructure components (e.g. light poles within the city streets) and configured to detect ambient air methane molecules at the city streets, and data communication to service center to broadcast ambient methane concentrations.

In another non-limiting example, disclosed sensors facilitate better measurements and better regulations. Accurate methane emission inventory is now a top priority of regulatory agencies in the US. For decades, industry relies on estimated emission factors for leak sources in oil fields. The drive to refine these data relies on development of high fidelity sensors and analytics methods to refine the “default” methane emission factors and replace the current estimates, thereby informing policy and industrial decision making for potential mitigation opportunities.

In an embodiment a method (e.g., for detecting one or more analytes of interest) is provided. The method includes receiving a stimulation waveform at a sensor. The stimulation waveform is applied to a sensing material of the sensor via at least one pair of electrodes in contact with the sensing material. The sensing material is in contact with an ambient environment. The method includes receiving an electrical signal at a controller circuit from the at least one pair of electrodes representative of an impedance response of the sensing material, and analyzing the impedance response of the sensing material at frequencies that provide a linear response of the sensing material to an analyte of interest and at least partially rejects effects of interferences.

Optionally, the method includes operating the sensor at a temperature of at least 50 degrees Celsius above an ambient temperature.

Optionally, the impedance response includes at least one of a real portion or imaginary portion.

Optionally, the analyzing of the impedance response includes identifying a frequency peak of the impedance response. Additionally or alternatively, the frequency peak is configured to be based on a characteristic of the analyte of interest.

In an embodiment a system (e.g., sensing system) is provided. The system includes a sensor having a sensing material and at least one pair of electrodes in contact with the sensing material, the sensing material configured to be in contact with an ambient environment. The system includes a controller circuit electrically coupled to the at least one pair of electrodes. The controller circuit is configured to generate a stimulation waveform for application to the sensing material of the sensor via the at least one pair of electrodes. The controller circuit is configured to receive an electrical signal from the at least one pair of electrodes representative of an impedance response of the sensing material, and analyze the impedance response of the sensing material at frequencies that provide a linear response of the sensing material to an analyte of interest and at least partially reject effects of interferences.

In an embodiment a method (e.g., for detecting one or more analytes of interest) is provided. The method includes receiving a stimulation waveform at a sensor. The stimulation waveform is applied to a sensing material of the sensor via at least one pair of electrodes in contact with the sensing material. The sensing material is in contact with an ambient environment. The method includes receiving an electrical signal at a controller circuit from the at least one pair of electrodes representative of an impedance response of the sensing material, and analyzing the impedance response of the sensing material at frequencies that provide a monotonic or non-monotonic response of the sensing material to an analyte of interest and at least partially reject effects of interferences.

Optionally, the system includes a heater configured to set a temperature of the sensor at a temperature of at least 50 degrees Celsius above an ambient temperature associated with the sensor.

Optionally, the impedance response includes at least one of a real portion or imaginary portion.

Optionally, the frequencies correspond to a real portion of the impedance response.

Optionally, the frequencies include a frequency peak of the impedance response. The controller circuit may be configured to analyze the impedance response by identifying the frequency peak. Additionally or alternatively, the frequency peak is configured to be based on a characteristic of the analyte of interest.

Optionally, the at least one pair of electrodes and sensing material are configured to be part of a non-resonant circuit.

Optionally, the at least one pair of electrodes and sensing material are configured to be part of an inductor capacitor resistor (LCR) circuit.

Optionally, the sensing material is a metal oxide. Additionally or alternatively, the metal oxide is a single-metal oxide, a perovskite oxide having two differently sized cations, or a mixed metal oxide composition.

Optionally, the sensing material is a semiconductor.

Optionally, the sensing material is configured to have the impedance response with a monotonic response.

Optionally, the sensing material is configured to reduce effects of humidity of the impedance response.

Optionally, the controller circuit is configured to utilize a principal component analysis to reduce effects of interferences and isolate the analyte of interest.

Optionally, the sensing material is configured to have a recovery time of the impedance response based on a frequency of the stimulation waveform.

Optionally, the sensor is positioned on a vehicle. The analyte of interest may represent ambient air pollution relative to the vehicle.

In an embodiment a method (e.g., for detecting one or more analytes of interest) is provided. The method includes receiving a stimulation waveform at a sensor. The stimulation waveform is applied to a sensing material of the sensor via at least one pair of electrodes in contact with the sensing material. The sensing material is in contact with an ambient environment. The method includes receiving an electrical signal at a controller circuit from the at least one pair of electrodes representative of an impedance response of the sensing material, and analyzing the impedance response of the sensing material at frequencies that provide a monotonic or non-monotonic response of the sensing material to an analyte of interest and at least partially reject effects of interferences.

As used herein, the terms “module”, “system,” “device,” “circuit,” or “unit,” may include a hardware and/or software system and circuitry that operates to perform one or more functions. For example, a module, unit, device, circuit, or system may include a computer processor, controller, or other logic-based device that performs operations based on instructions stored on a tangible and non-transitory computer readable storage medium, such as a computer memory. Alternatively, a module, unit, device, circuit, or system may include a hard-wired device that performs operations based on hard-wired logic and circuitry of the device. The modules, units, circuits, or systems shown in the attached figures may represent the hardware and circuitry that operates based on software or hardwired instructions, the software that directs hardware to perform the operations, or a combination thereof. The modules, systems, devices, circuits, or units can include or represent hardware circuits or circuitry that include and/or are connected with one or more processors, such as one or computer microprocessors.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the dimensions and types of materials described herein are intended to define the parameters of the inventive subject matter, they are by no means limiting and are exemplary embodiments. Many other embodiments will be apparent to one of ordinary skill in the art upon reviewing the above description. The scope of the inventive subject matter should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose several embodiments of the inventive subject matter, including the best mode, and also to enable one of ordinary skill in the art to practice the embodiments of inventive subject matter, including making and using any devices or systems and performing any incorporated methods. The patentable scope of the inventive subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

The foregoing description of certain embodiments of the present inventive subject matter will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (for example, processors or memories) may be implemented in a single piece of hardware (for example, a general purpose signal processor, microcontroller, random access memory, hard disk, or the like). Similarly, the programs may be stand alone programs, may be incorporated as subroutines in an operating system, may be functions in an installed software package, or the like. The various embodiments are not limited to the arrangements and instrumentality shown in the drawings.

As used herein, an element or step recited in the singular and proceeded with the word “a” or “an” should be understood as not excluding plural of said elements or operations, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “comprises,” “including,” “includes,” “having,” or “has” an element or a plurality of elements having a particular property may include additional such elements not having that property.

What is claimed is:

1. A method comprising:
receiving a stimulation waveform at a sensor, wherein the stimulation waveform is applied to a sensing material of the sensor via at least one pair of electrodes in contact with the sensing material, the sensing material being in contact with an ambient environment;
receiving an electrical signal at a controller circuit from the at least one pair of electrodes representative of an impedance response of the sensing material; and
analyzing the impedance response of the sensing material at frequencies that provide a linear response of the sensing material to an analyte of interest and at least partially rejects effects of interferences.

2. The method of claim 1, further comprising operating the sensor at a temperature of at least 50 degrees Celsius above an ambient temperature.

3. The method of claim 1, wherein the impedance response includes at least one of a real portion or imaginary portion.

4. The method of claim 1, wherein the analyzing of the impedance response includes identifying a frequency peak of the impedance response.

5. The method of claim 4, wherein the frequency peak is configured to be based on a characteristic of the analyte of interest.

6. A sensing system comprising:
a sensor having a sensing material and at least one pair of electrodes in contact with the sensing material, the sensing material configured to be in contact with an ambient environment; and
a controller circuit electrically coupled to the at least one pair of electrodes, the controller circuit is configured to generate a stimulation waveform for application to the sensing material of the sensor via the at least one pair of electrodes, the controller circuit configured to receive an electrical signal from the at least one pair of electrodes representative of an impedance response of the sensing material, and the controller circuit configured to analyze the impedance response of the sensing material at frequencies that provide a linear response of the sensing material to an analyte of interest and at least partially reject effects of interferences.

7. The sensing system of claim 6, further comprising a heater configured to set a temperature of the sensor at a temperature of at least 50 degrees Celsius above an ambient temperature associated with the sensor.

8. The sensing system of claim 6, wherein the impedance response includes at least one of a real portion or imaginary portion.

9. The sensing system of claim 6, wherein the frequencies correspond to a real portion of the impedance response.

10. The sensing system of claim 6, wherein the frequencies include a frequency peak of the impedance response, and the controller circuit is configured to analyze the impedance response by identifying the frequency peak.

11. The sensing system of claim 10, wherein the frequency peak is configured to be based on a characteristic of the analyte of interest.

12. The sensing system of claim 6, wherein the at least one pair of electrodes and sensing material are configured to be part of a non-resonant circuit.

13. The sensing system of claim 6, wherein the at least one pair of electrodes and sensing material are configured to be part of an inductor capacitor resistor (LCR) circuit.

14. The sensing system of claim 6, wherein the sensing material is a metal oxide.

15. The sensing system of claim 14, wherein the metal oxide is a single-metal oxide, a perovskite oxide having two differently sized cations, or a mixed metal oxide composition.

16. The sensing system of claim 6, wherein the sensing material is a semiconductor.

17. The sensing system of claim 6, wherein the sensing material is configured to have the impedance response with a monotonic response.

18. The sensing system of claim 6, wherein the sensing material is configured to reduce effects of humidity of the impedance response.

19. The sensing system of claim 6, wherein the controller circuit is configured to utilize a principal component analysis to reduce effects of interferences and isolate the analyte of interest.

20. The sensing system of claim 6, wherein the sensing material is configured to have a recovery time of the impedance response based on a frequency of the stimulation waveform.

21. The sensing system of claim 6, wherein the sensor is positioned on a vehicle, and the analyte of interest representing ambient air pollution relative to the vehicle.

22. A method comprising:
receiving a stimulation waveform at a sensor, wherein the stimulation waveform is applied to a sensing material of the sensor via at least one pair of electrodes in contact with the sensing material, the sensing material being in contact with an ambient environment;
receiving an electrical signal at a controller circuit from the at least one pair of electrodes representative of an impedance response of the sensing material; and
analyzing the impedance response of the sensing material at frequencies that provide a monotonic or non-monotonic response of the sensing material to an analyte of interest and at least partially reject effects of interferences.
